(19) United States
(12) Patent Application Publication
(10) Pub. No.: US 2023/0270352 A1
(43) Pub. Date: Aug. 31, 2023

(54) SYSTEM AND METHOD FOR MONITORING MUSCULOSKELETAL LOADING AND APPLICATIONS OF SAME

(71) Applicant: VANDERBILT UNIVERSITY, Nashville, TN (US)

(72) Inventors: Karl Zelik, Nashville, TN (US); Emily Matijevich, Nashville, TN (US); Peter Volgyesi, Nashville, TN (US)

(21) Appl. No.: 18/017,877

(22) PCT Filed: Jul. 29, 2021
(86) PCT No.: PCT/US2021/043631

§ 371 Date: Jan. 25, 2023

Related U.S. Application Data
(63) Continuation of application No. 17/051,218, filed on Oct. 28, 2020.
(60) Provisional application No. 63/058,066, filed on Jul. 29, 2020.
(60) Provisional application No. 63/124,961, filed on Dec. 14, 2020.
(60) Provisional application No. 62/664,479, filed on Apr. 30, 2018.

(51) Int. Cl. A61B 5/11; A61B 5/296; A61B 5/00

(52) U.S. Cl. CPC A61B 5/11; A61B 5/296; A61B 5/4509; A61B 5/4538; A61B 5/4806; A61B 5/6801; A61B 5/6898; A61B 5/7405; A61B 5/7435; A61B 5/7455; A61B 5/746; A61B 2562/0219; A61B 2562/0247

(57) ABSTRACT

A wearable device operably worn by a user for monitoring musculoskeletal loading on a back segment of the user includes a plurality of sensors that is synchronized to each other, each sensor operably attached at a predetermined location of the user and configured to detect information about a biomechanical activity of the musculoskeletal systems, wherein the plurality of sensors comprises at least one motion/orientation sensor; and a processing unit in communication with the plurality of sensors and configured to process the detected information by the plurality of sensors to estimate the musculoskeletal loading and/or damage and/or injury risk, and communicate the estimated musculoskeletal loading and/or damage and/or injury risk to the user and/or a party of interest.

### CROSS-REFERENCE TO RELATED PATENT APPLICATIONS

This application claims priority to and the benefit of U.S. Provisional Patent Application Ser. Nos. 63/058,066, filed Jul. 29, 2020, and 63/124,961, filed Dec. 14, 2020, which are incorporated herein by reference in their entireties.

This application is also a continuation-in-part application of U.S. patent application Ser. No. 17/051,218, filed Oct. 28, 2020, which is a national stage entry of PCT Patent Application Serial No. PCT/US2019/029790, filed Apr. 30, 2019, which itself claims priority to and the benefit of U.S. Provisional Patent Application Ser. No. 62/664,479, filed Apr. 30, 2018, which are incorporated herein by reference in their entireties.

### STATEMENT AS TO RIGHTS UNDER FEDERALLY-SPONSORED RESEARCH

This invention was made with government support under Contract No. R01EB028105 awarded by the National Institute of Biomedical Imaging and Bioengineering, and Contract No. K12HD073945 awarded by the Eunice Kennedy Shriver National Institute of Child Health and Human Development. The government has certain rights in the invention.

### FIELD OF THE INVENTION

This invention relates generally to biosensors, and more particularly, to wearable devices and methods to monitor musculoskeletal loading, estimate tissue microdamage and provide injury risk biofeedback and applications of the same.

### BACKGROUND OF THE INVENTION

The background description provided herein is for the purpose of generally presenting the context of the invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions. Work of the presently named inventors, to the extent it is described in the background of the invention section, as well as aspects of the description that may not otherwise qualify as prior art at the time of filing, are neither expressly nor impliedly admitted as prior art against the invention.

Low back disorders are a leading occupational health problem, ranging from lumbar (low back) pain to muscle strains to herniated spinal discs. Physical pain, missed work, decreased productivity, healthcare costs, short- and long-term disability, and psychological distress due to these low back disorders are substantial and persistent burdens on our society. Back disorders account for about 40% of all work-related musculoskeletal disorders, and about one in four workers reports dealing with low back pain. Individuals working in manual material handling jobs (and other jobs with similar physical demands) are at particularly high risk for low back disorders due to repetitive lifting and bending, which can lead to musculoskeletal overexertion (overuse) injuries.

Overexertion injuries result from an accumulation of microdamage caused by repetitive or sustained loading to musculoskeletal tissues (e.g., muscles, tendons, ligaments, bones, discs). Overexertion injuries are consistent with a fatigue failure process: the weakening and eventual failure of a material due to repeated loading. When modeling this fatigue failure process, both the number of loading repetitions and the magnitude of loading on the musculoskeletal tissues are important for approximating the cumulative damage to the tissues. Mechanical creep, which results from prolonged loading, can also be analyzed and included in models or estimates of microdamage. Microdamage refers to material or tissue damage formed at the microscopic level due to musculoskeletal loading, and may include microfractures, diffuse damage, and linear microcracks. Cumulative damage (or simply damage) refers to the accumulation of microdamage from repeated loading. If this cumulative damage becomes too large it can result in injury, pain, or impaired mechanical properties of musculoskeletal tissues. There are multiple opportunities to use non-invasive estimates of musculoskeletal loading (i.e., loading on one or more tissues inside the body, or surrogate metrics that are correlated with these tissue loads) along with fatigue or creep failure insights to understand and reduce the risk of overexertion injuries, such as through ergonomic assessments or continuous, personal monitoring of injury risk.

Ergonomic Assessments: Ergonomic risk assessment tools that evaluate low back loading and assess injury risk using fatigue failure principles have shown potential for predicting the incidence of low back disorders. For example, the Lifting Fatigue Failure Tool (LiFFT) estimates cumulative tissue damage to the low back using an estimate of lumbar moment. Cumulative damage across a series of lifting tasks estimated with LiFFT has been shown to explain 72-95% of the deviance in low back disorders from epidemiological databases. Ergonomic risk assessments are traditionally performed via direct observation by a trained professional. For instance, to perform an ergonomic assessment using LiFFT (or other assessment tools like the NIOSH Lifting Equation), an ergonomist or safety professional would monitor a single worker during their shift, or over a subset of representative job tasks, to manually record how much each lifted object weighed and how far away each lifted object was from the body, then input how many times each type of lift is performed during a shift. The time spent observing a worker depends on the variability of job tasks (e.g., short-vs. long-cycle jobs), but is often on the order of 1-8 h per job.

While these valuable ergonomic assessments and injury risk profiles can inform the use of ergonomic controls to minimize the risk to workers, the assessments can be time-consuming and costly. Assessments can become prohibitively expensive when there are a large variety of jobs at a given workplace or when job functions are remote, unobservable, highly variable, or infrequent. Moreover, this kind of time-intensive professional observation is impractical for personalized, continuous monitoring of injury risk over long durations or across an entire workforce. Video-based solutions that leverage advances in computer vision and machine learning have the potential to address some of these challenges by providing a semi-automated analysis of jobs. However, this approach is impractical for highly dynamic jobs (e.g., a construction worker moving all over a construction site), or jobs where visual obstructions occur (e.g., an aerial porter climbing in and out of arriving planes) and is not intended for personalized monitoring across an entire workforce. To efficiently evaluate ergonomic risk across a wide range of workers, high-risk jobs, and workplace environments, there remains a need for tools that enable the automated, unconstrained, non-invasive, and widespread monitoring of musculoskeletal loading and damage, particularly to the lower back.

Wearable Sensors at a Single Body Location for Ergonomic Assessment or Continuous Monitoring: Small, inexpensive, wearable sensors offer a promising solution for the unconstrained monitoring of job demands, including in confined spaces or during dynamic jobs. Wearable solutions could automate traditional job analysis or ergonomic assessments by replacing time-consuming observations and manual measurements with automated analytics from wearable sensor data, potentially improving the quality (e.g., consistency, accuracy) and quantity of data (e.g., the amount of assessment time per worker, the number of workers evaluated). Further, wearables can be practical for continuous monitoring, providing new opportunities to perform ergonomics assessments for remote and long-cycle duration jobs or for personalized, daily injury risk monitoring that could inform ergonomic controls. Continuous monitoring also has the long-term potential to usher in a new era of preventative occupational safety and health that transforms how musculoskeletal risk is managed and insured.

While wearable sensors offer an exciting tool for monitoring low back loading and overexertion risks, current commercial and research technologies have some key limitations. Current commercial products (e.g., StrongArm Fuse, Soter Analytics Clip&Go, Kinetic Reflex, and Modjoul Smartbelt) use a single inertial measurement unit (IMU) mounted on the waist, back, or chest and analyze motion data (e.g., trunk orientation or acceleration) and the frequency of lifting/bending. These types of devices are referred as wearable sensors at a single body location (or single wearable solutions, for short) in the disclosure, where this terminology is used because they each use hardware placed on one body location, although this hardware unit may contain multiple different sensors that measure numerous signals (e.g., IMUs are generally composed of accelerometers, gyroscopes, and magnetometers).

These single wearable solutions are relatively practical to implement in the workplace and may be most amenable to job analyses that characterizes postures and task frequency, but less well-suited for ergonomic assessments that quantitatively assess injury risk based on musculoskeletal loading and fatigue failure principles. This is because low back loading is dependent on factors beyond the kinematics of a single body segment, including the mass of the object being lifted and how far away the object is from the body. So, while these single wearable solutions can use segmental motion data to identify when a worker performs a deep forward bend, they are generally unable to distinguish, for instance, if the worker lifted a 5 lb box vs. a 45 lb box. The heavier mass in this example is expected to result in 65× more tissue damage (based on LiFFT, and assuming boxes are located 25 inches anterior to the lumbar spine). There are some use cases where single wearable solutions are expected to estimate low back loading fairly well (e.g., if the objects lifted are of known mass and are in a fairly consistent location relative to the body). However, there are cases where single wearable solutions are likely to be insufficient because they do not account for varying object masses, object locations, or other external forces on the body. In these cases, single wearable solutions could potentially provide inaccurate or misleading information about loading and cumulative damage to the low back, or unreliable insight on low back injury risk for a specific job, task, or worker.

Distributed Wearable Sensors for Ergonomic Assessment or Continuous Monitoring: Using multiple wearable sensors at distributed locations on the body has the potential to provide better estimates of low back loading by capturing and integrating additional dynamics data (e.g., body segment motions or orientations, forces or moments, muscle activity). These distributed sensor solutions are conceptually similar to what is done in motion analysis labs when data from cameras, force plates, and/or other measurement modalities are combined with biomechanical models to compute the loading on the back. An example of a commercial distributed wearable sensor system is the Xsens system that uses up to 17 IMUS on different body segments to track motion. These data can then be passed through analytics software (e.g., Scalefit, AnyBody) to estimate musculoskeletal loading on the back. However, similar to single sensor solutions, distributed IMU systems cannot automatically distinguish the mass of the object being lifted. Often, this additional information must be entered manually, or additional sensor modalities must be added, which increases the complexity of data collection and analysis. Thus, distributed IMU systems may only partially automate ergonomic assessments, or they may provide inaccurate estimates of low back loading if analytics software simply assume a default object mass.

To fully automate back load monitoring, several research studies suggest that adding force-instrumented shoes or pressure insoles along with distributed IMUS over the upper and/or lower body is promising. For instance, Faber et al. showed that by combining 8-17 IMUs and force-sensing shoes, lumbar moments could be estimated within 10-20% of peak extension moments. Conforti et al. found corroborating results, showing that with 12 IMUS and pressure insoles the peak axial load on the L5/S1 joint could be estimated with errors <5%. These systems work by trying to directly replicate lab-based biomechanical analysis; by tracking full-body or full lower-body dynamics (ground reaction forces and segmental motions) in order to apply inverse dynamics (physics-based) analysis. As a result a large number of sensor and sensor locations are needed, and it is important track motion of all (or nearly all) major body segments.

However, there are a couple of critical limitations of these approaches. First, many of these solutions were developed and evaluated on a limited range of manual material handling tasks. For instance, Faber et al. only evaluated four lifting tasks, all with a 10 kg box. It therefore remains unclear if these combinations of wearable sensors and/or algorithms are accurate and generalizable to a broad range of complex manual material handling motions, as performed in real world environments. Second, these wearable solutions require a large number of sensors distributed across the body, which introduces practical challenges related to technology implementation, ease of use, acceptance, and adoption. For scientific research or infrequent ergonomic assessment, the burden of distributed instrumentation may be an acceptable trade-off for increased accuracy. However, using numerous sensors requires longer donning and doffing times and more complexity, which presents a pragmatic barrier for workplace adoption. To enable more efficient and widespread ergonomic assessments or continuous monitoring of injury risk, there remains a need for a solution that requires a smaller number of wearable sensors (to be practical) and provides validated estimates of low back loading for a wide range of work-relevant tasks (to ensure accuracy). However, to achieve this requires a fundamentally different approach/system, which identifies and leverages a sparse sensor set and data fusion algorithms (e.g., using machine learning) that extend beyond inverse dynamics analysis, rather than attempting to replicate physics-based movement analysis commonly performed in research laboratories.

Therefore, a heretofore unaddressed need exists in the art to address the aforementioned deficiencies and inadequacies.

### SUMMARY OF THE INVENTION

In one aspect, the invention relates to a method for optimizing locations and numbers of wearable sensors for monitoring musculoskeletal loading of a user wearing the wearable sensors in manual material handling. According to the invention, the method and the wearable sensor device are capable of non-invasively estimating/monitoring loads on tissues inside the body or load metrics correlated with these tissue loads, and associated damage or injury risk.

The method in one embodiment includes providing a candidate set of wearable sensors, each wearable sensor placed on a body segment; synchronously collecting data from lab-based instrumentation and sensor signals from the wearable sensors across N participants each performing a range of tasks, such as manual material handling; developing wearable sensor algorithms using various combinations of the collected sensor signals as algorithm inputs and lab-based gold-standard estimates of musculoskeletal loading or damage or injury risk as an algorithm target, and identifying the most important types and locations of sensors for target estimation, and the number of sensors used for a predetermined algorithm estimation accuracy, by applying the wearable sensor algorithms to the wearable sensor signals.

In one embodiment, the lab-based gold-standard estimates of musculoskeletal loading are obtained by estimating ground reaction forces and kinematics based on the data collected from lab-based instrumentation and rigid-body inverse kinematics, and combining the ground reaction forces and kinematics via rigid-body inverse dynamics to obtain lumbar extension moments that are selected as a target musculoskeletal loading metric. Musculoskeletal loading metrics include for example forces, moments, stresses or strains on tissues inside the body, or surrogate metrics correlated with these tissue loads. Additional analysis steps such as musculoskeletal modeling or finite element modeling may be used to estimate one or more of these metrics, and these load metrics may in turn be used to estimate microdamage or injury risk metrics.

In one embodiment, said developing the wearable sensor algorithms is performed with a k-fold validation, and comprises using the sensor signals collected from (N−1) participants to train the algorithm; evaluating the algorithm accuracy on the sensor signals collected from the remaining participant, and repeating said using and said evaluating for all the N participants to yield wearable algorithm estimates of the lumbar extension moment for the entire dataset of all the candidate wearable sensor signals.

In one embodiment, said developing the wearable sensor algorithms further comprises developing reduced sensor algorithms using a reduced number of sensor signals for estimating musculoskeletal loading metrics such as lumbar extension moments, wherein the reduced number of sensor signals is corresponding to a reduced set of candidate sensors at one or more locations including trunk, pelvis, thigh, shank, and foot IMUs, and the pressure insoles.

In one embodiment, the algorithm workflow is repeated to develop the reduced sensor algorithms that each uses a reduced set of one (1) to five (5) sensor locations.

In one embodiment, said identifying the most important types and locations of sensors, and the number of sensors comprises computing the coefficient of determination (or other variability or accuracy metric) and relative wearable sensor signal importance to identify the most promising reduced sensor combinations and the most important sensors, respectively; evaluating wearable algorithm results using scatter plots and participant-specific results; and computing additional accuracy metrics to determine the performance and limitations of each sensor combination.

In one embodiment, when the coefficient of determination is greater than a threshold value, or maximized for a given number of sensor locations, then the sensor combinations are identified as the most promising reduced sensor combinations.

In one embodiment, the most promising reduced sensor combinations for accurately monitoring low back loading are a trunk IMU and pressure insoles.

In one embodiment, using signals from the trunk IMU and the pressure insoles together with a gradient boosted decision tree algorithm provides a practical, accurate, and automated way to monitor time series lumbar moments across the range of material handling tasks.

In one embodiment, the trunk IMU is replaceable by thigh IMUS, or a pelvis IMU, without compromising much accuracy.

In another aspect, the invention relates to a wearable device operably worn by a user for monitoring musculoskeletal loading on a back segment of the user. The wearable device in one embodiment comprises a plurality of sensors that is time-synchronized to each other, each sensor operably attached at a predetermined location of the user and configured to detect information about a biomechanical activity of the musculoskeletal system, a segment orientation, and/or a loading magnitude thereon, wherein the plurality of sensors comprises at least one motion/orientation sensor; and a processing unit in communication with the plurality of sensors and configured to process the detected information by the plurality of sensors to estimate the musculoskeletal loading and/or damage and/or injury risk, and communicate the estimated musculoskeletal loading and/or damage and/or injury risk to the user and/or a party of interest.

In one embodiment, the biomechanical activity of the musculoskeletal system comprises a segment orientation, a velocity or acceleration, and segmental load, wherein the segmental load comprises a location and/or magnitude of forces or moment applied to the body segment.

In one embodiment, the plurality of sensors further comprises at least one pressure-sensing insole operably worn on at least one foot of the user. The pressure-sensing insoles can be used to compute the force under the feet, which can then be analyzed to estimate or discern the weight of the object being lifted or handled. Object weight is a key biomechanical contributor to lumbar loading, and can be used in algorithms such as those summarized herein to help approximate target metrics of musculoskeletal loading on the back. The center of pressure of force under the feet, or other measurements derived from the pressure-sensing insoles, may also be used to supplement or enhance algorithm estimates of lumbar loading since the center of pressure tends to move away from the spine as a person bends further forward or an object is lifted further from the body.

In one embodiment, the at least one motion/orientation sensor comprises an IMU operably attached to the trunk, pelvis, thighs, or shanks of the user. For example, the IMU on the trunk can be used to estimate the orientation of the trunk, which is a key biomechanical contributor to lumbar loading, and can be used in algorithms to help approximate target metrics of musculoskeletal loading on the back. Other motion/orientation sensors include goniometers, inclinometers, encoder, string potentiometer, or sensors that measure relative motion between two or mode body segments (e.g., using radio frequency or other non-contact means).

In one embodiment, the at least one motion/orientation sensor comprises an IMU operably attached to the trunk or pelvis of the user, and two IMUs operably attached to the left and right thighs or shanks of the user.

In one embodiment, the at least one motion/orientation sensor further comprises two IMUs operably attached to a third location, e.g., the left and right feet of the user, and/or two IMUs operably attached to a fourth location, e.g., the left and right arms or hands of the user.

In one embodiment, the musculoskeletal loading comprises a lumbar moment or torque, a lumbar spine or disc force, and/or a muscle, muscle group, tendon or ligament force.

In one embodiment, the lumbar extension moment is used as a target musculoskeletal loading metric for estimating cumulative tissue damage to the low back using a fatigue failure and/or finite element analysis.

In one embodiment, the detected information by the plurality of sensors further includes information about the trunk or lumbar orientation/angles, or velocities, or accelerations, or frequency of lifting or bending movements, which are estimated or tracked, and then combined with or used in conjunction with the musculoskeletal loading estimates to estimate damage or assess injury risk.

In one embodiment, the wearable device is in communication with a separate sensing system that operably acquires data (e.g., size, weight, location) of items which the user lifts up or lays down and transmits the acquired data to the wearable device as an input, together with the detected information by the plurality of sensors, for processing the musculoskeletal loading and/or damage and/or injury risk.

In one embodiment, the separate sensing system comprises a warehouse management system (or other workplace or inventory management system) that tracks, monitors, or stores the weight, size or location of each object of which the user handles. For example, data from a single IMU (or other motion/orientation sensor) that monitors trunk orientation of the wearer can be combined with object weight data transmitted from the warehouse management system to estimate lumbar loading without requiring a pressure-sensing insole sensor. The warehouse management system would be synchronized with the wearable system, either using temporal or spatial (e.g., GPS) synchronization methods, and this could be done in real-time or in post-hoc analysis after data were collected. As one specific example, the trunk orientation (e.g., sagittal bending angle) estimate could be used as a surrogate for (or correlate of) the horizontal distance between a person's spine and object being lifted. This is because a person generally has to bend further forward to pick up or set down objects that are further away. A look-up table or regression equation can be developed to quantitatively relate trunk orientation to the estimated horizontal distance between the spine and object. Data from the synchronized warehouse management system (or other kind of workplace or inventory management system) would provide information on the weight of each object. These two streams of data are sufficient to estimate the peak load moment (a musculoskeletal loading metric for the back) by multiplying the weight of the object by the peak horizontal distance from the spine to object. The peak load moment for each lift could then be input to LiFFT, an existing ergonomics assessment tool to compute cumulative damage and injury risk to the low back. In a variation of this example, both the trunk orientation from the IMU and the physical location of the object in the warehouse management system could be combined as inputs into a single equation to estimate the horizontal distance from the spine to the object during lifting or lowering, or to compute other intermediate variables used to compute musculoskeletal loading, or as direct inputs into ergonomics assessment models (e.g., NIOSH Lifting Equation) to determine injury risk. In yet another variation of this example, data from an IMU and from an optical video camera (worn by the user, or positioned in the environment) could be combined into a single equation to estimate the horizontal distance from the spine to the object.

In one embodiment, the separate sensing system comprises a force-sensing crate handle or glove worn by the user that detects the force applied to each object of which the user handles.

In one embodiment, the detected information by the plurality of sensors is processed by statistical modeling.

In one embodiment, the statistical modeling comprises supervised model-based linear regression, decision trees or neural networks, and/or other data-driven machine learning or sensor fusion algorithms.

In one embodiment, the statistical modeling comprises a gradient boosted decision tree algorithm.

In one embodiment, the processing unit is further configured to estimate the musculoskeletal loading using reference data for calibrating or establishing a processing algorithm, wherein the reference data are either stored on data storage means in communication with the processing unit, or collected or inputted from a specific user.

In one embodiment, the reference data are obtained by lab-based sensors, and the data storage means comprises a database, a cloud storage system, and/or a computer readable memory.

In one embodiment, the processing unit is further configured to alert the user, via audio or vibrotactile feedback, when the musculoskeletal loading or microdamage accumulation or injury risk is greater than a threshold that is predetermined or a threshold that is calibrated for a specific user.

In one embodiment, the processing unit is further configured to advise the user on when and how to adjust their movements, actions or physical activity type and duration so as to reduce injury risks.

In one embodiment, the processing unit is further configured to communicate to a computer, a smartphone, a smartwatch, a tablet or other user feedback or data acquisition device for inputting user inputs, and outputting at least one of the estimated musculoskeletal loading, alert and advice, estimates of damage or damage accumulation, and/or probability of fracture or injury risk, and storing the estimated musculoskeletal loading, alert and advice, estimates of damage or damage accumulation, and/or probability of fracture or injury risk.

In one embodiment, the wearable device further comprises a biofeedback unit in communication with the processing unit for outputting and/or displaying at least one of the estimated musculoskeletal loading, alert and advice, estimates of damage or damage accumulation, and/or probability of fracture or injury risk using audible, visual, tactile, haptic, thermal, electrical or other biofeedback means, and storing the estimated musculoskeletal loading, alert and advice, estimates of damage accumulation, and/or probability of fracture or injury risk.

In one embodiment, the biofeedback unit comprises a user interface device for user inputs.

In one embodiment, the user inputs comprise height, weight, body mass index, age, gender, diet, training schedule, subjective pain/fatigue, bone cross-sectional area, bone geometry, bone density, bone composition, GPS position, altitude of the user and/or other personal health or demographic data.

In one embodiment, a reinforcement learning algorithm incrementally learns the optimal control of an assistive device from wearable sensor inputs based on real-time feedback from the user and previously observed motion trajectories.

In one embodiment, the information further comprises data acquired from additional sensors that monitor sleep patterns, heart rate, heart rate variability, rest time between physical activity or other markers of tissue rest or remodeling, or physiological recovery. These measures of rest or recovery can be included in more complex models of injury risk, which include both the processes that damage tissue (e.g., musculoskeletal loading) and processing enable tissues to recover or remodel.

In one embodiment, the damage is estimated by summing across load metrics taken to an exponential power.

In one embodiment, the plurality of sensor is combined with or integrated into an exoskeleton, exosuit, smart clothing, or other wearable assistance device.

In one embodiment, the plurality of sensor onboard the exoskeleton, exosuit, smart clothing or other wearable assistance device are used to estimate contributions to lumbar loading, and then these estimates are used in calculations of musculoskeletal loading and/or damage and/or injury risk on the back or other body segments, wherein the moment from the exoskeleton is subtracted from total lumbar moment to estimate moment borne by biological tissues.

In one embodiment, the musculoskeletal loading is used for control or evaluation of the exoskeleton, exosuit, smart clothing or other wearable assistance device. One example of how to use musculoskeletal loading for control is a powered exoskeleton controller designed to provide an assistance magnitude proportional to the amount of back loading (e.g., lumbar moment). Another example would be an exoskeleton controller that was designed to only assist the user once they surpassed a threshold of musculoskeletal loading, or cumulative damage, or injury risk. An example of how to use musculoskeletal loading for evaluation is that the exoskeleton moment contribution about the back can be subtracted out of the lumbar moment estimate to assess how much back relief the exoskeleton is providing, or how much exoskeleton assistance reduces musculoskeletal tissue damage or injury risk. This same evaluation method outlined above could be used for other body segments, by subtracting the device moment contribution about a body joint or segment from the total moment about that body joint or segment. The device moment contribution would either come directly from the assistance device itself (e.g., in the case of robotic devices that contain sensors and compute assistance levels on board) or from additional algorithms (e.g., combining segmental angle or motion data with a known stiffness of a spring in the assistance device to compute the moment generated by the spring under the associated amount of deflection).

In one embodiment, a state machine is used to identify specific activities, and then different algorithms are used to process information and to estimate the musculoskeletal loading and/or damage and/or injury risk depending on the current state.

In one embodiment, the estimates of the musculoskeletal loading and/or damage and/or injury risk are computed via real-time or near-real-time estimation algorithms.

In one embodiment, the estimated musculoskeletal loading and/or damage and/or injury risk is communicated to the user and/or a party of interest via one or more wireless or wired communication interfaces, either in real-time, near-real-time or at a later time.

In yet another aspect, the invention relates to a method for monitoring musculoskeletal loading on a back segment of a user wearing a wearable device including a plurality of sensors that is time-synchronized to each other, each sensor worn by the user at a predetermined location, wherein the plurality of sensors comprises at least one motion/orientation sensor. The method comprises receiving information about a biomechanical activity of the musculoskeletal system from the plurality of sensors; estimating musculoskeletal loading and/or damage and/or injury risk of the back segment based on the received information from the plurality of sensors; and communicating the estimated musculoskeletal loading and/or damage and/or injury risk to the user and/or a party of interest.

In one embodiment, the biomechanical activity of the musculoskeletal system comprises a segment orientation, a velocity or acceleration, and a segmental load, wherein the segmental load comprises a location and/or magnitude of force or moment applied to the body segment.

In one embodiment, the plurality of sensors further comprises at least one pressure-sensing insoles operably worn on at least one foot of the user. Alternatively, other types of force-measuring shoes or socks, or force-instrumentation between the foot and ground may be used.

In one embodiment, the at least one motion/orientation sensor comprises an inertial measurement unit (IMU) operably attached to the trunk, pelvis, thighs, or shanks of the user.

In one embodiment, the at least one motion/orientation sensor comprises a first IMU operably attached to the trunk or pelvis of the user, and two second IMUs operably attached to the left and right thighs or shanks of the user.

In one embodiment, the at least one motion/orientation sensor further comprises two IMUs operably attached to at a third location, e.g., the left and right feet of the user, and/or two IMUs operably attached to a fourth location, e.g., the left and right arms or hands of the user.

In one embodiment, the estimating step is performed by statistical modeling.

In one embodiment, the statistical modeling comprises supervised model-based linear regression, decision trees or neural networks, and/or other data-driven machine learning or sensor fusion algorithms.

In one embodiment, the statistical modeling comprises a gradient boosted decision tree algorithm.

In one embodiment, the estimating step computes the musculoskeletal loading using reference data to calibrate or establish the processing algorithm, so as to determine a condition of the body structure based on the estimated musculoskeletal loading, the condition including a normal condition or a graduated risk of injury.

In one embodiment, the reference data are obtained by motion analysis lab-based sensors.

In one embodiment, the communicating step comprises inputting user inputs, and outputting at least one of the estimated musculoskeletal loading, alert and advice, estimates of damage or damage accumulation, and/or probability of fracture or injury risk, and storing the estimated musculoskeletal loading, alert and advice, estimates of damage or damage accumulation, and/or probability of fracture or injury risk.

In one embodiment, the communicating step further comprises advising the user on when and how to adjust their movements, actions or physical activity type and duration so as to reduce injury risks.

In one embodiment, the plurality of sensors is combined with or integrated into an exoskeleton, exosuit, smart clothing, or other wearable assistance device.

In one embodiment, the method further comprises controlling or evaluating the exoskeleton, exosuit, smart clothing or other wearable assistance device using the musculoskeletal loading. In one embodiment, the control of the exoskeleton, exosuit, smart clothing or other wearable assistive device is optimized from wearable sensor inputs based on real-time feedback from the user and previously observed motion trajectories, using a reinforcement learning algorithm.

In one embodiment, the method further comprises identifying specific activities using a state machine, and processing information and to estimate the musculoskeletal loading and/or damage and/or injury risk with different algorithms depending on the current state.

In one embodiment, the estimates of the musculoskeletal loading and/or damage and/or injury risk are computed via real-time or near-real-time estimation algorithms.

In one embodiment, the estimated musculoskeletal loading and/or damage and/or injury risk is communicated to the user and/or a party of interest via one or more wireless or wired communication interfaces, either in real-time, near-real-time or at a later time.

In another aspect, the invention relates to a non-transitory computer-readable medium storing computer executable instructions to operate a wearable device for monitoring musculoskeletal loading on a back segment of a user according to the above disclosed method.

These and other aspects of the invention will become apparent from the following description of the preferred embodiment taken in conjunction with the following drawings, although variations and modifications therein may be affected without departing from the spirit and scope of the novel concepts of the invention.

### BRIEF DESCRIPTION OF THE DRAWINGS

The accompanying drawings illustrate one or more embodiments of the invention and, together with the written description, serve to explain the principles of the invention. Wherever possible, the same reference numbers are used throughout the drawings to refer to the same or like elements of an embodiment.

FIGS. 1A-1B show experimentation and wearable algorithm development overview, according to embodiments of the invention. FIG. 1A: Lab-based (green) and wearable sensor (orange) signals were collected synchronously in a motion analysis lab while participants performed about 400 manual material handling tasks. FIG. 1B: Lab-based analysis yielded a gold-standard estimate of lumbar extension moment (Mₑₓₜₑₙₛᵢₒₙ). Wearable signal analysis and algorithm development yielded wearable sensor estimates of lumbar extension moment (M′ₑₓₜₑₙₛᵢₒₙ). The wearable algorithm development was conducted twice, once using idealized wearable sensors (defined in Methods) as inputs (Analysis 1) and once using real wearable sensors as inputs (Analysis 2).

FIG. 2 shows maximum algorithm accuracy increased with number of sensor locations, according to embodiments of the invention. Average accuracy using idealized wearable sensors summarized here using the average coefficient of determination (r²) across all participants. Orange dots represent the distributed sensor algorithm (right) and the highest accuracy algorithms using 1 (left) and 2 (center) sensor locations. All the algorithms here were developed with idealized wearable sensor signals and the target was lumbar extension moment. A detailed summary of all the algorithm accuracies and the exact sensor combinations for each algorithm is included in FIG. 10.

FIG. 3 shows idealized wearable sensor signal importance, according to embodiments of the invention. Sagittal trunk angle and vertical GRFs are the most important signals for estimating lumbar moments. Signal importances are from the idealized wearable sensor algorithm for estimating lumbar extension moments. R=right; L=left.

FIGS. 4A-4B show algorithm accuracies for three idealized wearable sensor algorithms, according to embodiments of the invention. FIG. 4A: Lab-based (gold-standard) lumbar extension moment vs. idealized wearable algorithm estimates of lumbar moment for all time samples for an example participant (participant 8*). Positive moments correspond to lumbar extension moments. A line with a slope of one is added to visualize a perfect correspondence between lab-based and wearable estimates. BW×BH=body weight×body height. FIG. 4B: Coefficient of determination (r²) for each participant. Average results (avg, bottom) are equivalent to accuracies shown in FIG. 2. The trunk IMU algorithm was less accurate than the trunk IMU and pressure insoles algorithm, and then the distributed sensors algorithm (p<0.001 and p<0.001, respectively, based on Wilcoxon signed-rank test of the k-fold cross validation accuracy results). Comparing accuracies from the trunk IMU and pressure insoles algorithm vs. the distributed sensors algorithm yielded p=0.054. Average REVISE was converted into units of Nm (using mean participant height and weight) and included for reference.

FIG. 5 shows real wearable sensor signal importance, according to embodiments of the invention. Sagittal trunk angle and vertical GRFs are the most important signals for estimating lumbar moments, consistent with the findings from idealized wearable sensor analysis in FIG. 3. Signal importances here are from the real wearable sensor algorithm for estimating lumbar extension moments. R=right; L=left.

FIGS. 6A-6B show algorithm accuracies for three different real wearable sensor combinations, according to embodiments of the invention. FIG. 6A: Lab-based (gold-standard) lumbar moment vs. real wearable sensor algorithm estimates of lumbar moment for all time samples for an example participant (participant 8*). Positive moments correspond to lumbar extension moments. A line with a slope of one is added to visualize a perfect correspondence between lab-based and wearable estimates. BW×BH=body weight×body height. FIG. 6B: Coefficient of determination (r²) for each participant.

FIG. 7 shows a side-by-side comparison of algorithm performance using idealized versus real wearable sensor signals, according to embodiments of the invention. Gray lines are each of the 10 participants' accuracy results, and colored lines are the average (and standard deviation) across the 10 participants. Results using idealized wearable sensors are shown on the left (orange) and results using real wearable sensors are shown on the right (blue).

FIG. 8 shows examples of lumbar bending moment estimates based on different wearable sensor combinations, according to embodiments of the invention. Single IMU wearable does not capture key trends and peaks in lumbar loading when objects are lifted. Time-series lab-based lumbar extension moment (green) and idealized wearable algorithm moments developed with three different idealized wearable sensor combinations (orange). Shown is an example lifting task from the hundreds of manual material handling tasks performed for an example participant; 3 pick and place task cycles with a 10 kg box shown. While we only show a subset of time-series results here, we observed similar algorithm accuracy trends across the broad range of manual material handling tasks collected. Gray areas are approximately when the participant was holding the 10 kg box, white areas are when the participant had no object in their hands. The trunk IMU tends to perform worse when the box is being held or lifted, whereas the trunk IMU plus pressure insoles, and distributed sensors, are able to better track key lumbar loading trends (gray areas). BW×BH=body weight×body height.

FIG. 9 shows lumbar moment estimates based on different wearable sensor combinations, according to embodiments of the invention. Single IMU wearable does not capture increases in lumbar loading when heavier objects are lifted. Shown is an illustrative example from one participant: peak lumbar moment of squat tasks when increasing box masses are lifted (10 kg, 15 kg, 23 kg are shown). The trunk IMU and insole algorithm, and also the distributed sensor algorithm, captures the trend of increasing lumbar moment with heavier object mass. However, the trunk IMU algorithm does not; it predicts a similar peak moment with each lift regardless of the mass being lifted. A key difference and innovation is that configurations with insoles allow for an estimate of object weight, since the summing the pressure (or force) under each foot captures the weight on the person and the weight of the object they are handling. Object weight could alternatively be obtained by other means such as force-sensing gloves, force-instrumented object handles, or a warehouse management system that is preloaded with information about specific object weights. Analogous algorithms could be used with a single IMU (e.g., on the trunk) and these or other alternative estimates of object weight as inputs to the algorithm. BW×BH=body weight×body height.

FIG. 10 shows algorithm accuracies for all wearable sensor combinations, according to embodiments of the invention. Number of sensor locations and specific sensor combination influences algorithm accuracy. Top panel: Average accuracy using idealized wearable sensors summarized here using the average coefficient of determination (r²). Darker color bars correspond to an increasing number of sensor locations used in the algorithm. Bottom panel: Summary of which sensor locations were used in each reduced sensor combination. Orange grid boxes indicate that signals from that sensor location were used for the algorithm. These results are equivalent to FIG. 2 but depicted here to visualize the performance of each specific sensor combination.

FIG. 11 shows examples of lumbar lateral bending moment estimates based on different wearable sensor combinations, according to embodiments of the invention. Combining a trunk IMU and pressure insoles is also promising for estimating lumbar lateral bending moment. Average accuracy using idealized wearable sensors summarized here using the average coefficient of determination (r²). As with estimating lumbar extension moments (FIG. 2), the maximum algorithm accuracy increased with number of sensor locations. Orange dots represent the distributed sensor algorithm (6 sensor locations), and a subset of algorithms using 1 and 2 sensor locations. All the algorithms here were developed with idealized wearable sensor signals and the target was the lumbar lateral bending moment.

FIG. 12 shows examples of lumbar lateral bending moment estimates based on different wearable sensor combinations, according to embodiments of the invention. Shown are the lab-based (green) and algorithm-estimated (orange) lateral bending moments for three different sensor combinations. Depicted is a subset of the hundreds of manual material handling tasks performed for an example participant; 4 pick and place task cycles with a 5 kg box shown. These results were similar to those observed when estimating the lumbar extension moment (FIG. 8): the single IMU wearable did not well estimate the higher magnitude lateral bending moments, but combining a pressure insole with at least one IMU improved these estimates. All the algorithms here were developed with idealized wearable sensor signals and the target was lumbar lateral bending moment. BW×BH=body weight×body height.

FIG. 13 shows idealized wearable sensor signal importance, according to embodiments of the invention. Vertical GRFs and frontal trunk angle are the most important signals for estimating lumbar lateral bending moments. Signal importances are from the idealized wearable sensor algorithm for estimating the lumbar lateral bending moment. Note that these signals can be obtained from the same two sensors (trunk IMU and pressure insoles) that we identified as being the most important for estimating the lumbar extension moment. R=right; L=left.

FIG. 14 shows algorithm accuracies when estimating the lumbar lateral bending moment, according to embodiments of the invention. Shown are the participant-specific and average (avg) accuracy results from three different subsets of idealized wearable sensors: trunk IMU, trunk IMU and pressure insoles, and all distributed sensors. Accuracy is reported as the coefficient of determination (r²) across all time samples for a given participant.

FIGS. 15A-15B show idealized wearable sensor signal vs. idealized wearable sensor signals, according to embodiments of the invention. Trunk IMUs provided a relatively precise estimate of trunk orientation while pressure insoles provided a more variable estimate of vertical force. Ten scatter plots represent each participant and gray dots represent each time sample. Real wearable trunk orientation from an IMU correlated well with the idealized wearable trunk angle from lab-based motion capture (FIG. 15A), whereas the real wearable vertical force from pressure insoles did not correlate as well with the idealized wearable vertical force from lab-based force plates and exhibited a higher variability (FIG. 15B). A line with a slope of one is added to visualize a perfect correspondence between idealized and real wearable sensor signals. See Tables 1 and 2 for details on idealized and real wearable sensor signals. deg=degrees; BW=body weight.

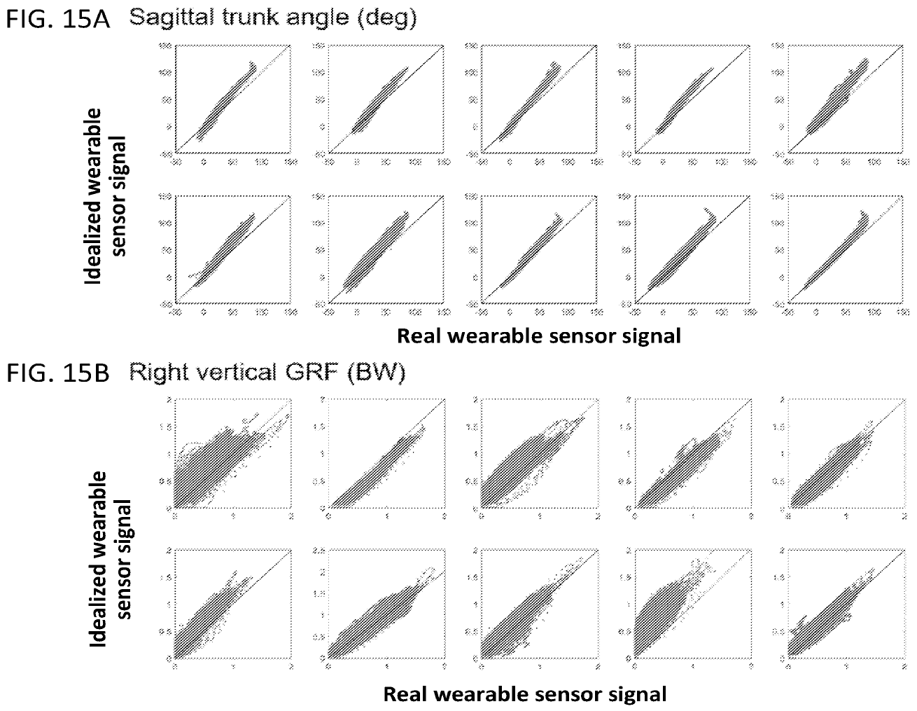

### DETAILED DESCRIPTION OF THE INVENTION

The invention will now be described more fully hereinafter with reference to the accompanying drawings, in which exemplary embodiments of the invention are shown. The invention may, however, be embodied in many different forms and should not be construed as limited to the embodiments set forth herein. Rather, these embodiments are provided so that this invention will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting and/or capital letters has no influence on the scope and meaning of a term; the scope and meaning of a term are the same, in the same context, whether or not it is highlighted and/or in capital letters. It will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only and in no way limits the scope and meaning of the invention or of any exemplified term. Likewise, the invention is not limited to various embodiments given in this specification.

It will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below can be termed a second element, component, region, layer or section without departing from the teachings of the invention.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” to another feature may have portions that overlap or underlie the adjacent feature.

The terminology used herein is for the purpose of describing particular embodiments only and is not intended to be limiting of the invention. As used herein, the singular forms “a”, “an”, and “the” are intended to include the plural forms as well, unless the context clearly indicates otherwise. It will be further understood that the terms “comprises” and/or “comprising,” or “includes” and/or “including” or “has” and/or “having” when used in this specification specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top” may be used herein to describe one element's relationship to another element as illustrated in the figures. It will be understood that relative terms are intended to encompass different orientations of the device in addition to the orientation shown in the figures. For example, if the device in one of the figures is turned over, elements described as being on the “lower” side of other elements would then be oriented on the “upper” sides of the other elements. The exemplary term “lower” can, therefore, encompass both an orientation of lower and upper, depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which the invention belongs. It will be further understood that terms, such as those defined in commonly used dictionaries, should be interpreted as having a meaning that is consistent with their meaning in the context of the relevant art and the present invention, and will not be interpreted in an idealized or overly formal sense unless expressly so defined herein.

As used herein, “around”, “about”, “substantially” or “approximately” shall generally mean within 20 percent, preferably within 10 percent, and more preferably within 5 percent of a given value or range. Numerical quantities given herein are approximate, meaning that the terms “around”, “about”, “substantially” or “approximately” can be inferred if not expressly stated.

As used herein, the terms “comprise” or “comprising”, “include” or “including”, “carry” or “carrying”, “has/have” or “having”, “contain” or “containing”, “involve” or “involving” and the like are to be understood to be open-ended, i.e., to mean including but not limited to.

As used in this invention, the phrase “at least one of A, B, and C” should be construed to mean a logical (A or B or C), using a non-exclusive logical OR. As used herein, the term “and/or” includes any and all combinations of one or more of the associated listed items.

The apparatuses and methods will be described in the following detailed description and illustrated in the accompanying drawings by various blocks, components, circuits, processes, algorithms, etc. (collectively referred to as “elements”). These elements may be implemented using electronic hardware, computer software, or any combination thereof. Whether such elements are implemented as hardware or software depends upon the particular application and design constraints imposed on the overall system. By way of example, an element, or any portion of an element, or any combination of elements may be implemented as a “processing system” that includes one or more processors. Examples of processors include microprocessors, microcontrollers, graphics processing units (GPUs), central processing units (CPUs), application processors, digital signal processors (DSPs), reduced instruction set computing (RISC) processors, systems on a chip (SoC), baseband processors, field programmable gate arrays (FPGAs), programmable logic devices (PLDs), state machines, gated logic, discrete hardware circuits, and other suitable hardware configured to perform the various functionality described throughout this disclosure. One or more processors in the processing system may execute software. Software shall be construed broadly to mean instructions, instruction sets, code, code segments, program code, programs, subprograms, software components, applications, software applications, software packages, routines, subroutines, objects, executables, threads of execution, procedures, functions, etc., whether referred to as software, firmware, middleware, microcode, hardware description language, or otherwise.

The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in different order (or concurrently) without altering the principles of the invention.

Low back disorders are a leading cause of occupational injury and physical disability, particularly in manual material handling, due to repetitive lumbar loading. Ergonomic assessments are often performed to understand and mitigate the risk of these musculoskeletal overexertion injuries. Wearable sensor solutions for monitoring low back loading have the potential to improve the quality, quantity, and efficiency of ergonomic assessments, and to expand opportunities for personalized, continuous monitoring of overexertion injury risk. However, current wearable solutions for monitoring low back injury risk either rely on metrics that are unreliable indicators of low back loading or require many distributed sensors that are impractical for implementation.

One of the objectives of the disclosure is to disclose a promising wearable solution for practical, automated, and accurate monitoring of low back loading using a small number of sensors, during manual material handling.

In one aspect, the invention relates to a method for optimizing locations and numbers of wearable sensors for monitoring musculoskeletal loading of a user wearing the wearable sensors in manual material handling. According to the invention, the method and the wearable sensor device are capable of non-invasively estimating/monitoring loads on tissues inside the body or load metrics correlated with these tissue loads, and associated damage or injury risk.

The method in one embodiment includes providing a candidate set of wearable sensors, each wearable sensor placed on a body segment; synchronously collecting data from lab-based instrumentation and sensor signals from the wearable sensors across N participants each performing a range of tasks, such as manual material handling; developing wearable sensor algorithms using various combinations of the collected sensor signals as algorithm inputs and lab-based gold-standard estimates of musculoskeletal loading or damage or injury risk as an algorithm target, and identifying the most important types and locations of sensors for target estimation, and the number of sensors used for a predetermined algorithm estimation accuracy, by applying the wearable sensor algorithms to the wearable sensor signals.

In some embodiments, the lab-based gold-standard estimates of musculoskeletal loading are obtained by estimating ground reaction forces and kinematics based on the data collected from lab-based instrumentation and rigid-body inverse kinematics, and combining the ground reaction forces and kinematics via rigid-body inverse dynamics to obtain lumbar extension moments that are selected as a target musculoskeletal loading metric. Musculoskeletal loading metrics include for example forces, moments, stresses or strains on tissues inside the body, or surrogate metrics correlated with these tissue loads. Additional analysis steps such as musculoskeletal modeling or finite element modeling may be used to estimate one or more of these metrics, and these load metrics may in turn be used to estimate microdamage or injury risk metrics.

In some embodiments, said developing the wearable sensor algorithms is performed with a k-fold validation, and comprises using the sensor signals collected from (N−1) participants to train the algorithm; evaluating the algorithm accuracy on the sensor signals collected from the remaining participant, and repeating said using and said evaluating for all the N participants to yield wearable algorithm estimates of the lumbar extension moment for the entire dataset of all the candidate wearable sensor signals.

In some embodiments, said developing the wearable sensor algorithms further comprises developing reduced sensor algorithms using a reduced number of sensor signals for estimating musculoskeletal loading metrics such as lumbar extension moments, wherein the reduced number of sensor signals is corresponding to a reduced set of candidate sensors at one or more locations including trunk, pelvis, thigh, shank, and foot IMUs, and the pressure insoles.

In some embodiments, the algorithm workflow is repeated to develop the reduced sensor algorithms that each uses a reduced set of one (1) to five (5) sensor locations.

In some embodiments, said identifying the most important types and locations of sensors, and the number of sensors comprises computing the coefficient of determination (or other variability or accuracy metric) and relative wearable sensor signal importance to identify the most promising reduced sensor combinations and the most important sensors, respectively; evaluating wearable algorithm results using scatter plots and participant-specific results; and computing additional accuracy metrics to determine the performance and limitations of each sensor combination.

In some embodiments, when the coefficient of determination is greater than a threshold value, or maximized for a given number of sensor locations, then the sensor combinations are identified as the most promising reduced sensor combinations.

In some embodiments, the most promising reduced sensor combinations for accurately monitoring low back loading are a trunk inertial measurement unit (IMU) and pressure insoles.

In some embodiments, using signals from the trunk IMU and the pressure insoles together with a gradient boosted decision tree algorithm provides a practical, accurate, and automated way to monitor time series lumbar moments across the range of material handling tasks.

In some embodiments, the trunk IMU is replaceable by thigh IMUS, or a pelvis IMU, without compromising much accuracy.

In another aspect, the invention relates to a wearable device operably worn by a user for monitoring non-invasively estimating/monitoring loads on tissues inside the body or load metrics correlated with the tissue loads, and associated damage or injury risk on a back segment of the user. The wearable device in one embodiment comprises a plurality of sensors that is time-synchronized to each other, each sensor operably attached at a predetermined location of the user and configured to detect information about a biomechanical activity of the musculoskeletal system, wherein the plurality of sensors comprises at least one motion/orientation sensor; and a processing unit in communication with the plurality of sensors and configured to process the detected information by the plurality of sensors to estimate the musculoskeletal loading and/or damage and/or injury risk, and communicate the estimated musculoskeletal loading and/or damage and/or injury risk to the user and/or a party of interest.

In some embodiments, the biomechanical activity of the musculoskeletal system comprises a segment orientation, a velocity or acceleration, and a segmental load, wherein the segmental load comprises a location and/or magnitude of force or moment applied to the body segment.

In some embodiments, the plurality of sensors further comprises at least one pressure-sensing insoles operably worn on at least one foot of the user. The pressure-sensing insoles can be used to compute the force under the feet, which can then be analyzed to estimate or discern the weight of the object being lifted or handled. Object weight is a key biomechanical contributor to lumbar loading, and can be used in algorithms such as those summarized herein to help approximate target metrics of musculoskeletal loading on the back. The center of pressure of force under the feet, or other measurements derived from the pressure-sensing insoles, may also be used to supplement or enhance algorithm estimates of lumbar loading since the center of pressure tends to move away from the spine as a person bends further forward or an object is lifted further from the body.

In some embodiments, the at least one motion/orientation sensor comprises an IMU operably attached to the trunk, pelvis, thighs, or shanks of the user. For example, the IMU on the trunk can be used to estimate the orientation of the trunk, which is a key biomechanical contributor to lumbar loading, and can be used in algorithms to help approximate target metrics of musculoskeletal loading on the back. Other motion/orientation sensors include goniometers, inclinometers, encoders, string potentiometers, GPS, or sensors that measure relative motion between two or mode body segments (e.g., using radio frequency or other non-contact means).

In some embodiments, the at least one motion/orientation sensor comprises an IMU operably attached to the trunk or pelvis of the user, and two IMUs operably attached to the left and right thighs or shanks of the user.

In some embodiments, the at least one motion/orientation sensor further comprises two IMUs operably attached to the left and right feet of the user, and/or two IMUs operably attached to the left and right arms or hands of the user.

In one embodiment, the plurality of sensors includes one IMU (e.g., attached to the trunk or pelvis of the wearer/user) and one pressure insole (e.g., worn by a foot of the user). In another embodiments, the plurality of sensors includes two to five IMUs and one pressure insole. In yet another embodiment, the plurality of sensors includes one IMU only. In this case, data of objects of which the user handles acquired by a load metric from separate system (e.g., warehouse management system) are used, together with the information from the IMU for non-invasively estimating/monitoring loads on tissues inside the body or load metrics correlated with the tissue loads, and associated damage or injury risk. In a further embodiment, the plurality of sensors includes two pressure insoles and one trunk IMU. In an alternative embodiment, the plurality of sensors includes one or two pressure insoles, one trunk IMU or one pelvis IMU, and/or thigh IMU.

In some embodiments, the musculoskeletal loading comprises a lumbar moment or torque, a lumbar spine or disc force, and/or a muscle, muscle group, tendon or ligament force.

In some embodiments, the lumbar extension moment is used as a target musculoskeletal loading metric for estimating cumulative tissue damage to the low back using a fatigue failure and/or finite element analysis.

In some embodiments, the detected information by the plurality of sensors further includes information about the trunk or lumbar orientation/angles, or velocities, or accelerations, or frequency of lifting or bending movements, which are estimated or tracked, and then combined with or used in conjunction with the musculoskeletal loading estimates to estimate damage or assess injury risk.

In some embodiments, the wearable device is in communication with a separate sensing system that operably acquires data (e.g., size, weight, location) of objects which the user lifts up or lays down and transmits the acquired data to the wearable device as an input, together with the detected information by the plurality of sensors, for processing the musculoskeletal loading and/or damage and/or injury risk.

In some embodiments, the separate sensing system comprises a warehouse management system (or other kind of workplace or inventory management system) that tracks the size, weight, or location of each object of which the user handles. For example, a single IMU that monitors trunk orientation of the user can be combined with object weight transmitted from the warehouse management system to estimate lumbar loading without requiring a pressure-sensing insole sensor. The warehouse management system would be synchronized with the wearable system, either using temporal or spatial (e.g., GPS) synchronization methods, and this could be done in real-time or in post-hoc analysis after data were collected. Accurate tracking of objects allows for estimates of how much a worker must bend to lift an object at a given location and how the much object weighed, such that these could then be used to enhance wearable sensor estimates of musculoskeletal loading, or used to compute an independent estimate of musculoskeletal loading, damage, and/or injury risk.

In some embodiments, the separate sensing system comprises a force-sensing crate handle or glove worn by the user that detects the force applied to each object of which the user handles.

A key difference and innovation is that configurations with insoles allow for an estimate of object weight, since summing the pressure (or force) under each foot captures the body weight of the person and the weight of the object they are handling. Object weight could alternatively be obtained by other means such as force-sensing gloves, force-instrumented object handles, or a warehouse management system that is preloaded with information about specific object weights, or combinations thereof. Analogous algorithms could be used with a single IMU (e.g., on the trunk) and these alternative estimates of object weight as inputs to the algorithm.

In some embodiments, the detected information by the plurality of sensors is processed by statistical modeling.

In some embodiments, the statistical modeling comprises supervised model-based linear regression, decision trees or neural networks, and/or other data-driven machine learning or sensor fusion algorithms.

In some embodiments, the statistical modeling comprises a gradient boosted decision tree algorithm.

In some embodiments, the processing unit is further configured to estimate the musculoskeletal loading using reference data for calibrating or establishing a processing algorithm, wherein the reference data are either stored on data storage means in communication with the processing unit, or collected or inputted from a specific user.

In some embodiments, the reference data are obtained by lab-based sensors, and the data storage means comprises a database, a cloud storage system, and/or a computer readable memory.

In some embodiments, the processing unit is further configured to alert the user, via audio or vibrotactile feedback, when the musculoskeletal loading or cumulative damage or injury risk is greater than a threshold that is predetermined or a threshold that is calibrated for a specific user.

In some embodiments, the processing unit is further configured to advise the user on when and how to adjust their movements, actions or physical activity type and duration so as to reduce injury risks.

In some embodiments, the processing unit is further configured to communicate to a computer, a smartphone, a smartwatch, a tablet or other user feedback or data acquisition device for inputting user inputs, and outputting at least one of the estimated musculoskeletal loading, alert and advice, estimates of damage or damage accumulation, and/or probability of fracture or injury risk, and storing the estimated musculoskeletal loading, alert and advice, estimates of damage or damage accumulation, and/or probability of fracture or injury risk.

In some embodiments, the wearable device further comprises a biofeedback unit in communication with the processing unit for outputting and/or displaying at least one of the estimated musculoskeletal loading, alert and advice, estimates of damage or damage accumulation, and/or probability of fracture or injury using audible, visual, tactile, haptic, thermal, electrical or other biofeedback means, and storing the estimated musculoskeletal loading, alert and advice, estimates of damage accumulation, and/or probability of fracture or injury risk.

In some embodiments, the biofeedback unit comprises a user interface device for user inputs.

In some embodiments, the user inputs comprise height, weight, body mass index, age, gender, diet, training schedule, subjective pain/fatigue, bone cross-sectional area, bone geometry, bone density, bone composition, GPS position, altitude of the user and/or other personal health or demographic data.

In some embodiments, a reinforcement learning algorithm incrementally learns the optimal control of an assistive device from wearable sensor inputs based on real-time feedback from the user and previously observed motion trajectories.

In some embodiments, the information further comprises data acquired from additional sensors that monitor sleep patterns, heart rate, heart rate variability, rest time between physical activity or other markers of tissue rest or remodeling, or physiological recovery. These measures of rest or recovery can be included in more complex models of injury risk, which include both the biomechanical processes that damage tissue (e.g., musculoskeletal loading) and physiological processes that enable tissues to recover or remodel.

In some embodiments, the damage is estimated by summing across load metrics taken to an exponential power.

In some embodiments, the plurality of sensor is combined with or integrated into an exoskeleton, exosuit, smart clothing, or other wearable assistance device.

In some embodiments, the plurality of sensor onboard the exoskeleton, exosuit, smart clothing or other wearable assistance device are used to estimate contributions to lumbar loading, and then these estimates are used in calculations of musculoskeletal loading and/or damage and/or injury risk on the back or other body segments, wherein the moment from exoskeleton is subtracted from total lumbar moment to estimate moment borne by biological tissues.

In some embodiments, the musculoskeletal loading is used for control or evaluation of the exoskeleton, exosuit, smart clothing or other wearable assistance device. One example of how to use musculoskeletal loading for control is a powered exoskeleton controller designed to provide an assistance magnitude proportional to the amount of back loading (e.g., lumbar moment). Another example would be an exoskeleton controller that was designed to only assist the user once they surpassed a threshold of musculoskeletal loading, or cumulative damage, or injury risk. An example of how to use musculoskeletal loading for evaluation is that the exoskeleton moment contribution about the back can be subtracted out of the lumbar moment estimate to assess how much back relief the exoskeleton is providing, or how much exoskeleton assistance reduces musculoskeletal tissue damage or injury risk. This same evaluation method outlined above could be used for other body segments, by subtracting the device moment contribution about a body joint or segment from the total moment about that body joint or segment. The device moment contribution would either come directly from the assistance device itself (e.g., in the case of robotic devices that contain sensors and compute assistance levels on board) or from additional algorithms (e.g., combining segmental angle or motion data with a known stiffness of a spring in the assistance device to compute the moment generated by the spring under the associated amount of deflection).

In some embodiments, a state machine is used to identify specific activities, and then different algorithms are used to process information and to estimate the musculoskeletal loading and/or damage and/or injury risk depending on the current state.

In some embodiments, the estimates of the musculoskeletal loading and/or damage and/or injury risk are computed via real-time or near-real-time estimation algorithms.

In some embodiments, the estimated musculoskeletal loading and/or damage and/or injury risk is communicated to the user and/or a party of interest via one or more wireless or wired communication interfaces, either in real-time, near-real-time or at a later time.

In yet another aspect, the invention relates to a method for monitoring musculoskeletal loading on a back segment of a user wearing a wearable device including a plurality of sensors that is time-synchronized to each other, each sensor worn by the user at a predetermined location, wherein the plurality of sensors comprises at least one motion/orientation sensor. The method comprises receiving information about a biomechanical activity of the musculoskeletal system from the plurality of sensors; estimating musculoskeletal loading and/or damage and/or injury risk of the back segment based on the received information from the plurality of sensors; and communicating the estimated musculoskeletal loading and/or damage and/or injury risk to the user and/or a party of interest.

In some embodiments, the biomechanical activity of the musculoskeletal system comprises a segment orientation, a velocity or acceleration, and a segmental load, wherein the segmental load comprises a location and/or magnitude of force or moment applied to the body segment.

In some embodiments, the plurality of sensors further comprises at least one pressure-sensing insoles operably worn on at least one foot of the user. Alternatively, other types of force-measuring shoes or socks, or force-instrumentation between the foot and ground may be used.

In some embodiments, the at least one motion/orientation sensor comprises an IMU operably attached to the trunk, pelvis, thighs, or shanks of the user.

In some embodiments, the at least one motion/orientation sensor comprises a first IMU operably attached to the trunk or pelvis of the user, and two second IMUs operably attached to the left and right thighs or shanks of the user.

In some embodiments, the at least one motion/orientation sensor further comprises two third IMUs operably attached to the left and right feet of the user, and/or two fourth IMUs operably attached to the left and right arms or hands of the user.

In some embodiments, the estimating step is performed by statistical modeling.

In some embodiments, the statistical modeling comprises supervised model-based linear regression, decision trees or neural networks, and/or other data-driven machine learning or sensor fusion algorithms.

In some embodiments, the statistical modeling comprises a gradient boosted decision tree algorithm.

In some embodiments, the estimating step computes the musculoskeletal loading using reference data to calibrate or establish the processing algorithm, so as to determine a condition of the body structure based on the estimated musculoskeletal loading, the condition including a normal condition or a graduated risk of injury.

In some embodiments, the reference data are obtained by motion analysis lab-based sensors.

In some embodiments, the communicating step comprises inputting user inputs, and outputting at least one of the estimated musculoskeletal loading, alert and advice, estimates of damage or damage accumulation, and/or probability of fracture or injury risk, and storing the estimated musculoskeletal loading, alert and advice, estimates of damage or damage accumulation, and/or probability of fracture or injury risk.

In some embodiments, the communicating step further comprises advising the user on when and how to adjust their movements, actions or physical activity type and duration so as to reduce injury risks.

In some embodiments, the plurality of sensors is combined with or integrated into an exoskeleton, exosuit, smart clothing, or other wearable assistance device.

In some embodiments, the method further comprises controlling or evaluating the exoskeleton, exosuit, smart clothing or other wearable assistance device using the musculoskeletal loading. In one embodiment, the control of the exoskeleton, exosuit, smart clothing or other wearable assistive device is optimized from wearable sensor inputs based on real-time feedback from the user and previously observed motion trajectories, using a reinforcement learning algorithm.

In some embodiments, the method further comprises identifying specific activities using a state machine, and processing information and to estimate the musculoskeletal loading and/or damage and/or injury risk with different algorithms depending on the current state.

In some embodiments, the estimates of the musculoskeletal loading and/or damage and/or injury risk are computed via real-time or near-real-time estimation algorithms.

In some embodiments, the estimated musculoskeletal loading and/or damage and/or injury risk is communicated to the user and/or a party of interest via one or more wireless or wired communication interfaces, either in real-time, near-real-time or at a later time.

In one or more example embodiments, the method and algorithms and functions described may be implemented in hardware, software, or any combination thereof. If implemented in software, the method and algorithms and functions may be stored on or encoded as one or more instructions or code on a non-transitory computer-readable medium, such that, when the one or more instructions or code are executed by one or more processors, the execution of the one or more instructions or code causes the wearable device to perform a method for musculoskeletal loading on a back segment of a user wearing the wearable device. The non-transitory computer-readable media includes computer storage media. Storage media may be any available media that can be accessed by a computer. By way of example, and not limitation, such computer-readable media can comprise a random-access memory (RAM), a read-only memory (ROM), an electrically erasable programmable ROM (EEPROM), optical disk storage, magnetic disk storage, other magnetic storage devices, combinations of the aforementioned types of computer-readable media, or any other medium that can be used to store computer executable code in the form of instructions or data structures that can be accessed by a computer.

These and other aspects of the present invention are further described below. Without intent to limit the scope of the invention, examples and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### Example

### Promising Wearable Solution for the Practical and Accurate Monitoring of Low Back Loading in Manual Material Handling

Low back disorders are a leading cause of missed work and physical disability in manual material handling due to repetitive lumbar loading and overexertion. Ergonomic assessments are often performed to understand and mitigate the risk of musculoskeletal overexertion injuries. Wearable sensor solutions for monitoring low back loading have the potential to improve the quality, quantity, and efficiency of ergonomic assessments and to expand opportunities for the personalized, continuous monitoring of overexertion injury risk. However, existing wearable solutions using a single inertial measurement unit (IMU) are limited in how accurately they can estimate back loading when objects of varying mass are handled, and alternative solutions in the scientific literature require so many distributed sensors that they are impractical for widespread workplace implementation.

Therefore, one of the objectives of this exemplary study is to explore new ways to accurately monitor low back loading using a small number of wearable sensors.

Based on the review of commercial technologies and scientific literature and the conversations and observations with manual material handlers and safety professionals across a range of industries (e.g., logistics, manufacturing, retail, agriculture, construction, military), a key technological gap and unmet industry need related to ergonomic assessment and continuous personal monitoring of low back overexertion injury risk were identified. Specifically, a portable wearable sensor tool was developed with the following characteristics and capabilities that does not exist in the current wearable solutions, which could be game-changing for low back injury risk assessment, monitoring, and prevention in various industries:
1. The tool is practical to don, doff, and wear in unconstrained environments for prolonged periods of time by virtue of using only a small number of sensors at different body locations. This is important for industrial acceptance, adoption, and implementation. Of note, there is no simple limit for the maximum number of sensors or body locations that is practical, but this consideration helped motivate the approach in this study, as detailed in the section of MATERIALS AND METHODS below.
2. The tool provides accurate, validated, and automated estimates of low back loading for a broad range of manual material handling tasks. This is important to ensure the system is reliable during use in the real world and can distinguish differences in back loading that result from lifting objects of different weights without the need for professional observation or manually inputting object weights or other data.

The overarching question the exemplary study sought to address was: if only a small number of wearable sensors can be used to monitor low back loading, then which sensors should be used, where should the sensors be placed, what type of algorithms should be employed to fuse the sensor data, and how accurately can low back loading be monitored during manual material handling tasks? To address this exploratory, multi-faceted, open-ended question, we collected synchronized data from laboratory instrumentation and wearable sensors across a broad range of lifting tasks and combined domain expertise in biomechanics with techniques from machine learning to develop musculoskeletal load estimation algorithms, similar to the approach we previously took to develop a wearable sensor system for monitoring bone loading and overexertion injury risks in the legs of runners.

We found that the two key sensors for accurately monitoring low back loading are a trunk IMU and pressure insoles. Using signals from these two sensors together with a Gradient Boosted Decision Tree algorithm has the potential to provide a practical (relatively few sensors), accurate (up to r²=0.89), and automated way (using wearables) to monitor time series lumbar moments across a broad range of material handling tasks. The trunk IMU could be replaced by thigh IMUs, or a pelvis IMU, without sacrificing much accuracy, but there was no practical substitute for the pressure insoles. The key to realizing accurate lumbar load estimates with this approach in the real world will be optimizing force estimates from pressure insoles.

### Materials and Methods

Summary of Approach: The exploratory approach is summarized herein, followed by detailed methodology below:

First, a candidate set of wearable sensors (number, type, and location of sensors) was identified. The candidate sensors were bounded based on biomechanical insight, prior literature, and expected practicality for implementation in the real world. IMUs placed on body segments (feet, shanks, thighs, pelvis, and trunk) and pressure insoles placed inside the shoes (capable of estimating the interaction force and center of pressure between the foot and shoe) were selected as the candidate sensors. These types of sensors are mature, and for years have been used in clinical and consumer devices that are worn daily; for instance, IMUs are ubiquitous in fitness trackers and phones, and pressure insoles are used for clinical screening (e.g., Orpyx) and to track running/sport performance (e.g., ARION, ReTiSense, NURVV). In this exemplary study, we elected not to use surface electromyography (EMG) due to practical challenges of implementing in the real world, such as their sensitivity to sweat, hair, and sensor placement, and reliability issues over days/weeks. We also elected not to use any implantable or percutaneous sensors, or any emerging sensor technologies that have not yet been proven to be practical, reliable, affordable, and scalable in the real world. Focusing on mature, proven sensor technologies was with the hope and intention of arriving at a solution that would be feasible to translate into a product for real world use in the near future (e.g., next 2-5 years).

Second, data from lab-based instrumentation and from real wearable sensors were synchronously collected across 10 participants each performing about 400 different manual material handling tasks, which encompassed many different postures, movements, and object masses that a worker may encounter in the real world.

Third, wearable sensor algorithms was developed using various combinations of wearable sensor signals (algorithm inputs) and the lab-based gold-standard estimates of low back loading (algorithm target). First, idealized wearable sensor signals, which included lab-based data converted into the types of signals reasonably obtained with wearables, were used to develop and evaluate algorithms. An example of an idealized wearable sensor signal is that the three-dimensional ground reaction force (GRF) vector from an in-ground force plate was mapped onto a one-dimensional force normal to the bottom of the foot to represent the type of signal that can be estimated from a pressure insole. This allowed us to explore algorithms for low back load estimation without worrying if the sensor or signal quality from a particular wearable sensor was a limiting factor. Next, real wearable sensor signals were used to separately develop and evaluate algorithms, benchmark the accuracy of current wearable sensor technologies, and assess how these may or may not limit low back load monitoring tools. In the disclosure, the terminology idealized wearable sensor signals and real wearable sensor signals are used to distinguish these two complementary approaches. Throughout, the terms idealized wearable sensors and real wearable sensors are also used to refer to physical sensors or sensor combinations, with idealized wearable sensors referring to the sensors that would be needed to measure the particular signals used. See the Discussion below for more rationale on the value of using idealized wearable sensor signals when exploring novel solutions for musculoskeletal load monitoring.

Finally, by applying various machine learning techniques to various subsets of idealized and real wearable sensor signals, we: (1) quantified how the number of sensors used influenced the algorithm estimation accuracy, (2) identified the most important types and locations of sensors for low back load estimation, and (3) benchmarked how much using real vs. idealized wearable sensor signals influenced the estimation accuracy. Below, we describe the human participant experiment and data analysis, followed by algorithm exploration, development, and evaluation.

Experiment Overview: In the exemplary study, ten healthy individuals participated in the study: 3 females and 7 males (age: 25±3 years; height: 1.8±0.1 m; mass: 79±14 kg). All the participants gave written informed consent to the protocol, which was approved by the Institutional Review Board at Vanderbilt University (IRB #141697).

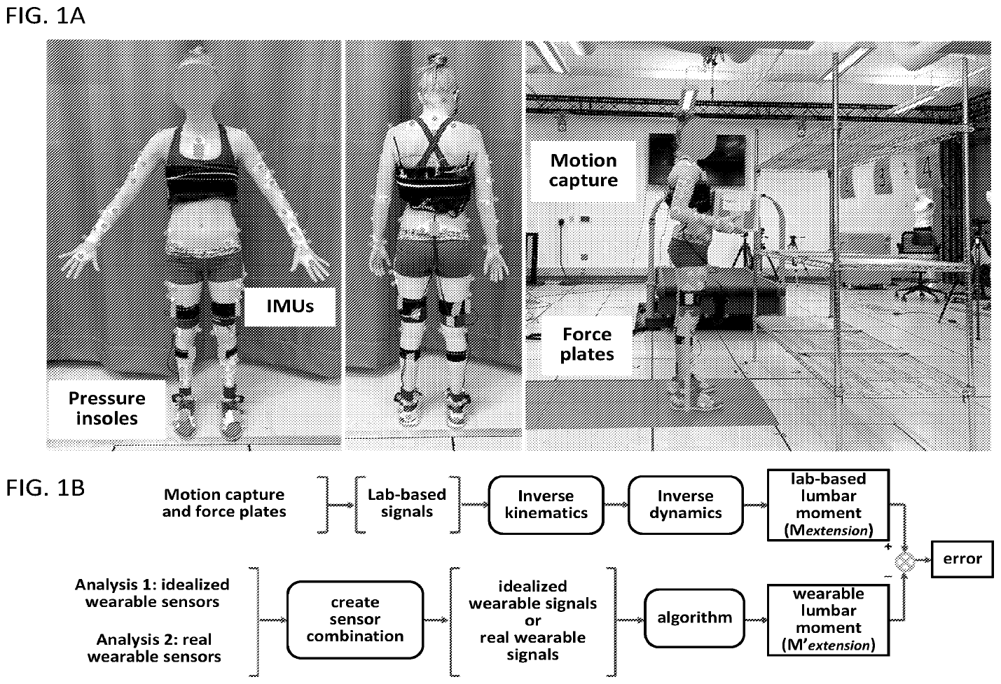

The study involved participants each performing about 400 manual material handling tasks in a motion analysis lab. Tasks covered a broad range of bending, turning, twisting, squatting, stooping, and reaching postures while lifting and moving boxes of 5-23 kg, which were representative of tasks commonly performed by manual material handlers (e.g., case pickers in a warehouse, retail workers stocking shelves, or logistics workers at a sort facility). For instance, tasks involved moving boxes from high to low shelves, low to high shelves, from a lateral to a forward position, diagonally between shelves, and much more to obtain a rich, diverse, realistic, and work-relevant data set. The data collection space was outfitted with various shelves at 3 heights with labeled locations (see FIG. 1A for an example setup). Box masses, shelf heights, and actions were informed by manual lifting and ergonomics guidelines. For each task, participants were given instructions such as “move the box from position 3 to 4” (FIG. 1A) and were told to use any safe strategy to complete the task. Each task was performed once and the participants were given rest breaks intermittently throughout the protocol.

A. Lab-Based Measurement Modalities: Full-body kinematics and ground reaction forces (GRFs) were collected. Kinematics were collected at 200 Hz (Vicon), then low pass-filtered at 6 Hz (3rd order, zero-lag Butterworth). Four markers were placed on each thigh, shank, arm, and forearm; 5 markers were placed on each foot; 6 markers were placed on the pelvis; and 4 were placed on the trunk. Additional markers were placed on the lateral and medial femoral epicondyles, the lateral and medial malleoli, each acromion, the lateral and medial humeral epicondyles, and the distal radius and ulna. The GRFs under each foot were collected at 1000 Hz using in-ground force plates (AMTI). The GRFs were low pass-filtered at 10 Hz (3rd order, zero-lag Butterworth).

B. Wearable Measurement Modalities: IMU-based lower body and trunk kinematics (Xsens) and plantar pressures (Novel pedar-x, with 99 pressure sensors per insole) were synchronously collected. Kinematics were collected at 100 Hz using the standard Xsens “lower body+trunk” configuration and IMUS were oriented according to the Xsens participant preparation guidelines. Scaling, calibration, and data pre-processing were performed by the Xsens software, providing a built-in anatomical model. Plantar pressures were collected bilaterally at 100 Hz and the total (normal) force and center of pressure were exported using the Novel software. The synchronization of all measurement modalities was achieved through recorded analog triggers, and any delays between measurement modalities were accounted for through temporal alignment/calibration algorithms based on pilot testing.

Wearable Algorithm Development: A visual overview of the lab-based data analysis and algorithm evaluation workflow is provided in FIG. 1B.

A. Lab Based Data Analysis (Algorithm Target): Lumbar extension moment was selected as the target musculoskeletal loading metric because it can be used to estimate cumulative tissue damage to the low back using a fatigue failure analysis. We sought to estimate the time series lumbar extension moment (as opposed to just peak moments) because this enables us to identify bending/lifting frequency, to partition out individual movement cycles, and to better understand and distinguish cyclic lifts vs. prolonged bending. Time series data enables the assessment of loading and cumulative risk across all tasks, as well as the ability to perform task-specific load and risk assessment.

Lower-body segmental and joint kinematics were estimated based on optical motion capture data and rigid-body inverse kinematics. GRF and kinematics were combined via rigid-body inverse dynamics to estimate joint kinetics (C-Motion, Visual3D). Time series lab-based lumbar moment was estimated using bottom-up inverse dynamics in Visual3D. Lumbar extension moments are reported in units of body weight×body height (BW×BH).

B. Wearable Sensor Signal Data Preparation (Algorithm Inputs): Time series wearable sensor signals were used as inputs to the algorithm. Idealized wearable sensor signals are summarized in Table 1. Real wearable sensor signals are summarized in Table 2. The algorithm development workflow was completed twice, once using idealized wearable sensor signals as the inputs and once using real wearable sensor signals as the inputs (Analysis 1 and Analysis 2, FIG. 1B). The lab-based target, idealized wearable sensor signals, and real wearable sensor signals were all resampled to 100 Hz. Input signals were normalized to z-scores during the algorithm development.

TABLE 1
Idealized Wearable Sensor Signals, R = right, L = left.

| | | Number |
|---|---|---|
| | Idealized Wearable | of |
| Idealized Wearable Sensors | Sensor Signals | Signals |
| 8 idealized IMUs (trunk, pelvis, R/L | XYZ segment kinematics | 24 |
| thigh, R/L shank. R/L foot) | (Euler angles) | |
| Segments (8): pelvis, trunk, R/L | XYZ joint kinematics | 21 |
| thigh, R/L shank. R/L foot | | |
| Joints (7): lumbar, R/L hip, R/L | | |
| knee, R/L ankle | | |
| Idealized pressure insoles (R/L) | 3D force plate GRF | 2 |
| | transformed into foot's | |
| | coordinate frame and | |
| | projected onto 1D normal | |
| | force | |
| | Force plate center of | 4 |
| | pressure transformed | |
| | into foot's X/Y | |
| | coordinate frame | |

| | |
|---|---|
| Total: | 51 |

TABLE 2
Real Wearable Sensor Signals, R = right, L = left.

| | Idealized Wearable | # of |
|---|---|---|
| Idealized Wearable Sensors | Sensor Signals | Signals |
| 8 IMUs (sternum, pelvis, R/L thigh, | XYZ segment kinematics | 33 |
| R/L shank. R/L foot) | (Euler angles) | |
| Segments (11): pelvis, L5, L3, T12, | Segment kinematics | 44 |
| T8, R/L thigh, R/L shank. R/L foot | (quaternions) | |
| Joints (10): L5S1, L4L3, L1T12, | XYZ segment velocities | 33 |
| T9T8, R/L hip, R/L knee, R/L ankle | XYZ segment | 33 |
| | accelerations | |
| | XYZ joint kinematics | 30 |
| Pressure insoles (R/L) | Total normal force | 2 |
| | X/Y center of pressure | 4 |

| | |
|---|---|
| Total: | 179 |

C. Algorithm Development: We explored supervised machine learning algorithms (e.g., generalized linear models, support vector machines, neural networks) for multiple variable regression to predict the lumbar extension moment (Mₑₓₜₑₙₛᵢₒₙ) focusing on techniques that could provide instantaneous predictions, where wearable signals from a given time sample are used to estimate the target load metric for that same time sample. Ultimately, the most promising results were achieved with Gradient Boosted Decision Trees, a popular technique in machine learning and well-suited to handle missing values and redundant or non-predictive inputs. The number of input signals (tens or hundreds) also fits this approach. Furthermore, by using a histogram-based decision tree building algorithm influenced by LightGBM, the algorithm training time was dramatically decreased (to a few seconds with a few million time samples) without a noticeable degradation in the prediction accuracy. Briefly, this algorithm estimates the target load metric by building an ensemble of decision trees in a stage-wise fashion, where in each stage the new tree tries to estimate (and thus, remove) the residual error after combining the predictions of the previous trees. The current results are based on ensembles of approximately 100 trees. We used the scikit-learn library and Amazon SageMaker, a cloud-based machine learning platform for algorithm development, model training, and evaluation. It should be appreciated that other libraries and platforms may also be used to practice the invention.

To develop the algorithm, we used k-fold validation by participant (S=10), a commonly used technique to assess the generalizability of an algorithm. In other words, we used data from nine participants to train the algorithm (i.e., select hyperparameters), and then evaluated the algorithm accuracy on data from the remaining participant. This process was repeated for all ten participants to yield wearable algorithm estimates of the lumbar extension moment (M′ₑₓₜₑₙₛᵢₒₙ) for the entire dataset.

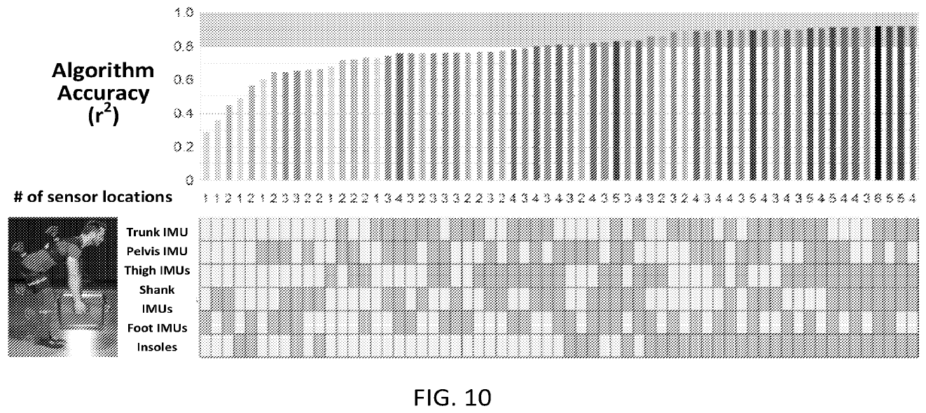

The algorithm workflow was first performed using all the candidate wearable sensor signals; which is termed as the distributed sensor algorithm. Next, to evaluate the feasibility of using a reduced number of sensors for estimating lumbar moments, we developed additional algorithms using a reduced number of sensor signals (termed reduced sensor algorithms). While we explored 10 candidate wearable sensors (R/L pressure insoles, R/L foot IMUs, R/L shank IMUs, R/L thigh IMUs, pelvis IMU, trunk IMU), when iterating through potential reduced sensor algorithms, we assumed that a final solution would have symmetrical bilateral sensors (e.g., if the wearable included a right insole, then it would also include a left insole). Thus, the 10 candidate wearable sensors actually corresponded to 6 candidate sensor locations: trunk, pelvis, thigh, shank, and foot IMUs, and the pressure insoles. The algorithm workflow was repeated to develop 62 additional algorithms that each used a reduced set of 1 to 5 sensor locations (see FIG. 10 for an overview of all the combinations).

D. Algorithm Evaluation: the accuracy of different sensor combinations was evaluated in two stages. First, we computed the coefficient of determination (r²) to identify the most promising reduced sensor combinations and computed relative wearable sensor signal importance to identify the most important sensors. Then, we identified promising or interesting sensor combinations, reviewed wearable algorithm results using scatter plots and participant-specific results, and computed additional accuracy metrics to better understand the performance and limitations of each sensor combination.

We computed r² for each participant across all time samples for all candidate sensor combinations. Based on our prior work on wearables for musculoskeletal load monitoring, we have found r² to be useful for this initial sensor combination selection process (i.e., down selection from 62 sensor combinations here) because it provides insight into how well wearable estimates correlate with lab-based gold-standard estimates across the full range of lumbar moments observed. This research is early stage, so there is no precise r² threshold that we can define as the minimum viable, but to benchmark high algorithm accuracy we used r²>0.8 as a threshold for promising solutions.

As a complementary analysis to evaluate which sensors were most important for algorithm estimates, we applied the permutation feature importance method. Feature importance values represent the drop in model accuracy (Δr²) when an input signal is randomly shuffled, with larger values indicating that the algorithm is more dependent on that signal. Of note, the permutation feature importance method was used rather than the impurity-based feature importance approach because the latter approach had some undesirable biases (e.g., favoring high cardinality features) and is not supported with histogram-based estimators.

Once a subset of promising sensor combinations was identified, we inspected participant-specific results with scatter plot data to understand the performance and limitations of each. We were particularly interested in how each sensor combination performed across the range of lumbar moment magnitudes observed (e.g., did certain sensor combinations perform better at low magnitudes vs. high magnitudes). We also computed the root mean square error (RMSE). In this data set, most samples are at relatively low lumbar moment magnitudes, but larger moments are the most damaging and dangerous to musculoskeletal tissues. We therefore also looked specifically at algorithm performance constrained to higher lumbar moments using mean absolute percent error (MAPE). We leveraged the benefits of both relative (r² and MAPE) and absolute (RMSE) accuracy metrics, along with biomechanics knowledge of key factors that influence cumulative damage and overexertion injury risk, to make informed suggestions about using wearable sensors to monitor back loading across work-relevant lifting tasks.

### Results

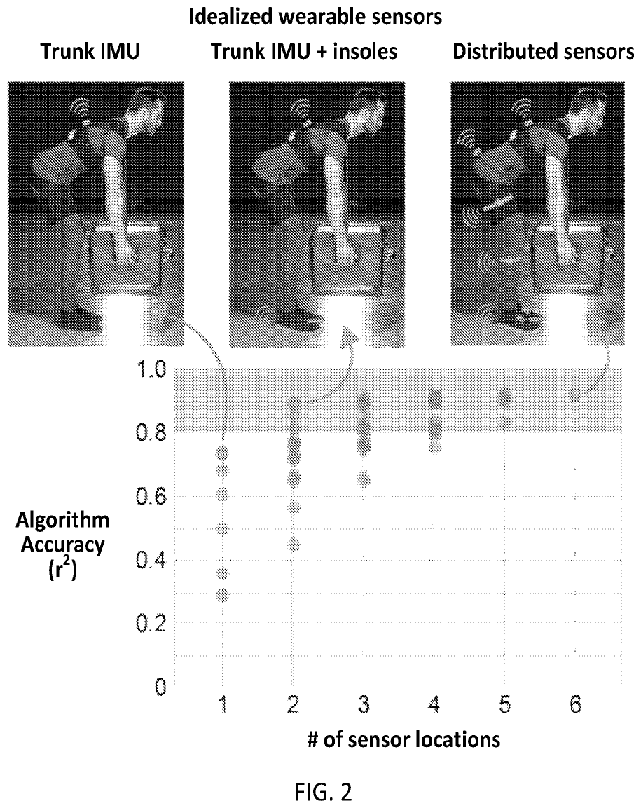

Results from Idealized Wearable Sensors: As expected, the maximum algorithm accuracy increased with the number of sensor locations, as shown in FIG. 2. There were no single sensor solutions that yielded r²>0.8 (i.e., coefficient of determination greater than 0.8 between idealized wearable sensor algorithm estimates and lab-based lumbar moment estimates). However, there was a noticeable jump in accuracy when moving from one to two sensor locations (maximum r²=0.74 to r²=0.89, FIG. 2, Table 3). When increasing the number of sensors beyond two locations, there were only small additional improvements in the maximum algorithm accuracy (from r²=0.89 using two sensor locations to r²=0.92 using all six sensor locations, the maximum number of distributed sensor locations in this study).

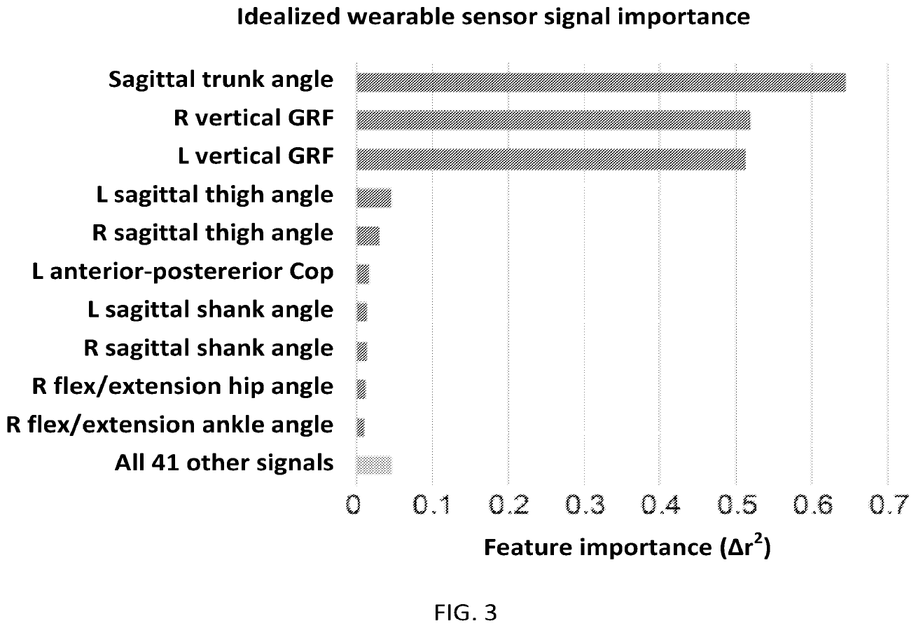

The two most important signals for estimating lumbar extension moments identified during algorithm development were sagittal trunk angle and vertical GRFs, as show in FIG. 3. Consistent with this, the best solution using two sensor locations is the one that combined a trunk IMU and pressure insoles (r²=0.89, FIG. 2, Table 3). This combination was of highest interest to us because of its potential to be practical and accurate.

TABLE 3
Algorithm accuracy for a subset of idealized wearable algorithms.
Average accuracy for the distributed sensor algorithm and the
top three algorithms requiring one or two sensor locations. Accuracies
reported here correspond to data points in FIGS. 2 and 10.

| Number of | Idealized Wearable | Algorithm |
|---|---|---|
| Sensor Locations | Sensor Combination | Accuracy (r²) |
| 6 | Distributed sensors | 0.92 |
| 2 | Trunk IMU + insoles | 0.89 |
| 2 | Thigh IMUs + insoles | 0.86 |
| 2 | Pelvis IMU + insoles | 0.81 |
| 1 | Trunk IMU | 0.74 |
| 1 | Thigh IMUs | 0.68 |
| 1 | Pelvis IMU | 0.61 |

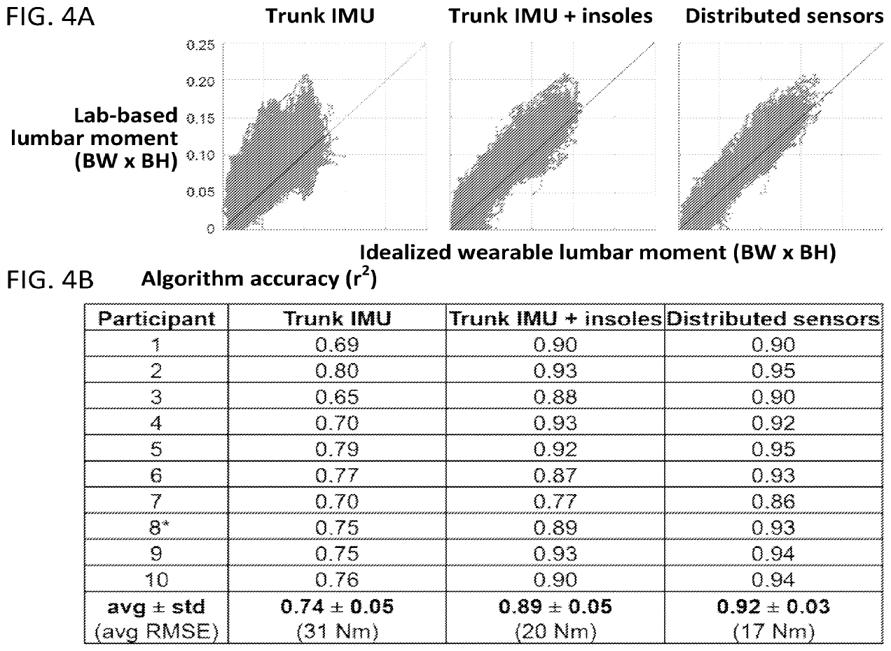

The trunk IMU (alone) and fully distributed sensor set were also of interest for further analysis. The trunk IMU provides a point of reference for the potential accuracy of existing commercial wearables that use a single IMU to monitor lumbar loading, while the distributed sensor set provides insight on accuracy gains with higher instrumentation coverage. Therefore, we report participant-specific results and additional accuracy summary metrics (RMSE and MAPE) for these different sensor combinations, as shown in FIGS. 4A-4B.

The distributed sensor algorithm resulted in an average RMSE of approximately 17 Nm (FIG. 4B), equivalent to about a 241 N (0.3 BW) error in spine compression force (assuming a 7 cm lumbar extensor muscle moment arm). The trunk IMU and pressure insole algorithm resulted in an average RMSE of approximately 20 Nm, equivalent to about a 282 N (0.4 BW) error in spine compression force. The trunk IMU algorithm resulted in an average RMSE error of approximately 31 Nm, equivalent to about a 444 N (0.6 BW) error in spine compression force. As one additional point of reference, the NIOSH Lifting Equation recommends limiting spine compression force to less than 3400 N (4.4 BW), so these RMSE values are about 7%, 9%, and 14% of this limit, respectively. Given the sensitivity of MAPE when target values are close to zero, we also computed the MAPE for all samples when the target load metric was greater than 0.05 BW×BH (which encompassed about half of all time samples of data for each participant). The average MAPE for the upper range of lumbar moments was 13%, 15%, and 25% for the distributed sensor, trunk IMU and pressure insole, and trunk IMU algorithms, respectively.

We also observed that if the trunk IMU were substituted with thigh IMUs, then correlations only decreased slightly from r²=0.74 to r²=0.68 with a single sensor, and from r²=0.89 to r²=0.86 for the two sensor combination (Table 3). If the trunk IMU were substituted with a pelvis IMU, then the correlations decreased slightly more from r²=0.74 to r²=0.61 with a single sensor, and from r²=0.89 to r²=0.81 for the two sensor combination (Table 3). All of the two sensor location solutions that achieved r²>0.8 included GRFs from pressure insoles.

Participant-specific results (FIGS. 4A-4B) corroborated and strengthened the average results (FIGS. 2 and 3, Table 3). For instance, all ten participants exhibited high algorithm accuracies (r² ranging from 0.86 to 0.95) using the distributed (six sensor location) algorithm. When moving from a single trunk IMU to using a trunk IMU and pressure insoles, every participant exhibited an increase in r² value (FIGS. 4A-4B). Scatter plot data for each participant indicated that the improvement in r² going from one to two sensor locations was driven by both a decrease in the variation of data about the unity regression line and improved estimates at higher magnitude lumbar moments (see the example participant data in FIG. 4A). When moving from two to six sensor locations, the variation in data about the regression line decreased more, but only slightly (FIG. 4A). We also note that for two participants (numbers 1 and 4), going from two to six sensor locations did not increase r² at all (FIG. 4B).

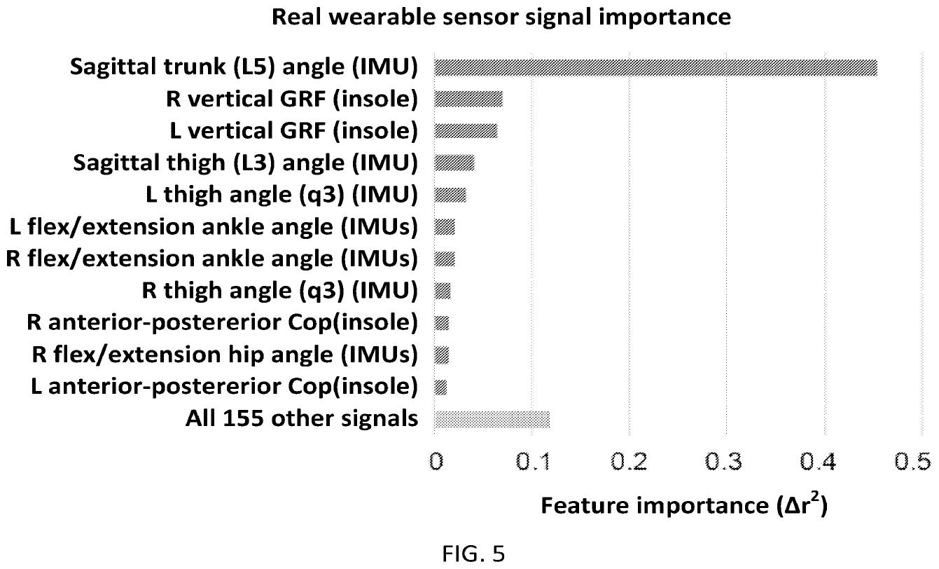

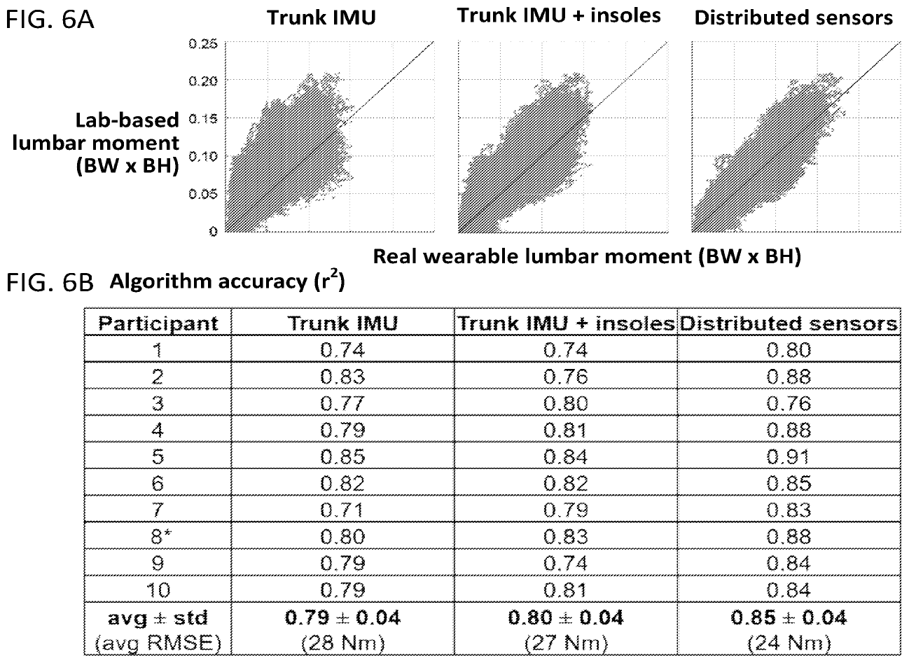

Results from Real Wearable Sensors: FIG. 5 is analogous to FIG. 3, and FIGS. 6A-6B is analogous to FIGS. 4A-4B, except that FIGS. 5 and 6A-6B are based on algorithms using real wearable sensors rather than idealized wearable sensors. Real wearable sensor results confirm that the most important sensor signals for estimating lumbar extension moments are sagittal trunk angle from a trunk IMU and vertical GRFs from pressure insoles (FIG. 5). However, it is noteworthy that the trunk angle signal importance was much higher than the vertical GRFs in the analysis using real wearable sensors (FIG. 5), whereas with idealized signals these signal importances were of similar magnitude (FIG. 3).

The participant-specific results (FIGS. 6A-6B) again corroborated and strengthened the average results from real wearable sensor algorithms. Compared to idealized wearable sensor algorithms, there was no discernible increase in r² value when moving from one sensor location (trunk IMU, r²=0.79) to two sensors locations (trunk IMU and pressure insoles, r²=0.80). The increase in r² from two to six sensor locations also remained relatively small, similar to what was observed in the idealized wearable sensor analysis (FIGS. 4A-4B).

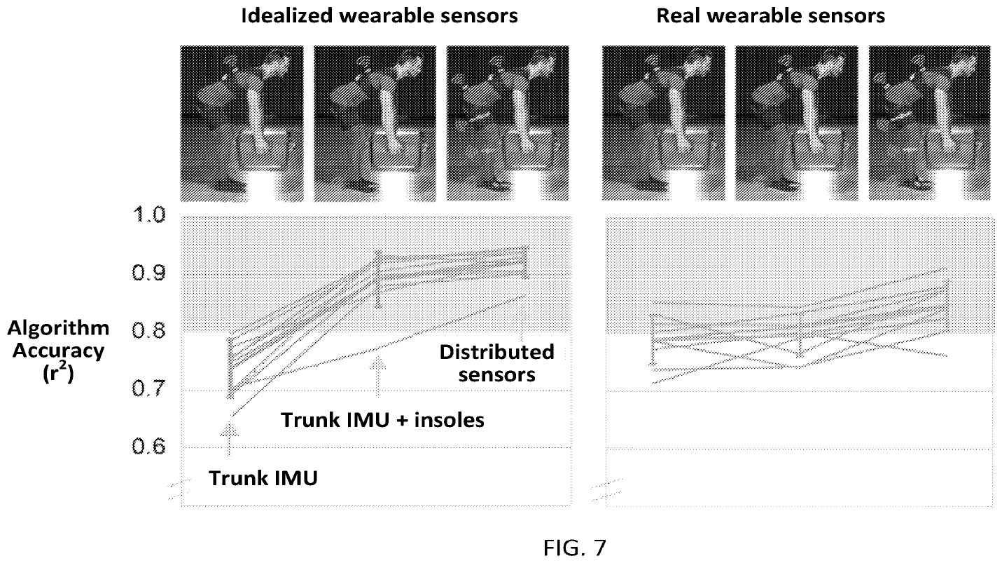

Comparison of Results from Idealized versus Real Wearable Sensors: FIG. 7 provides a side-by-side comparison of algorithm performance using idealized versus real wearable sensor signals. These plots are visualizations of the tabular results reported in FIGS. 4B and 6B and provided for clarity and to assist with interpretation. The key takeaway is that while the idealized wearable sensor analysis resulted in a noticeable jump in accuracy when moving from one to two sensors, a similar improvement was not observed in the real wearable sensor analysis (FIG. 7). The Discussion section below digs into why.

### Discussion

These findings indicate that there is strong potential to use a small number of wearable sensors to create a portable tool for the practical and accurate monitoring of low back loading over a broad range of manual material handling tasks. We characterized the performance of over 60 different wearable sensor combinations and algorithms. The solution we found to be most promising combines signals from sensors at two body locations (an IMU on the trunk and pressure insoles under the feet) with a Gradient Boosted Decision Tree algorithm. While idealized wearable sensor results demonstrated promising proof-of-concept, the analysis of real wearable sensor signals revealed that to achieve accurate lumbar moment estimates in the real world, the key technological challenge will be to optimize force estimates and minimize variability from the pressure insoles. With further development and validation, we believe that this type of wearable solutions has the potential to transform how ergonomic assessments are performed in industry, to enhance the quality, quantity, and efficiency of occupational data collection, and to expand opportunities for personalized, continuous monitoring of low back injury risk. For example, time-series lumbar moments could be partitioned into individual lift/bend cycles and the magnitude and frequency of loading on the low back could be automatically input into ergonomic assessment tools like LiFFT to estimate overexertion injury risk. Below we discuss the major technical findings from this exploratory research, along with alternative solutions, key challenges, and new opportunities for advancement.

Which Wearable Sensors and Locations Are Most Important? The trunk IMU and pressure insoles were identified in all analyses as together being the most important sensors for monitoring lumbar extension moments, as shown in FIGS. 2, 3 and 5, and Table 3. These results match our biomechanics intuition given that lumbar moment is strongly influenced by the weight of the object being lifted, which can be captured by pressure insoles, and by upper-body posture, which can be estimated with an IMU on the trunk.

Interestingly, the trunk IMU could be replaced with thigh IMUS or a pelvis IMU with relatively little degradation in accuracy, as shown in Table 3. Of note, the reason that the thigh and pelvis IMU signals appear to have a low importance shown in FIGS. 3 and 5, but can actually be useful substitutes for the trunk IMU is because they are highly correlated with other signals and because of how the feature importance method works (see Methods). It is valuable to acknowledge these other alternatives because some sensor locations may be preferred for certain applications; for instance, a fall protection harness manufacturer may be able to integrate an IMU more easily on the trunk near the D-ring or on the thighs using the leg loops, whereas for a tool belt manufacturer it may be preferable to integrate the IMU at the waist. In contrast, there was no substitute for the pressure insoles, which provide unique force data that helps to distinguish if the person is lifting a heavy object vs. a light object vs. no object at all and just bending forward. In theory, object mass could be obtained using sensors beyond those we tested (e.g., measured directly using force-instrumented gloves, or estimated indirectly via muscle EMG), but these again introduce added complexity and practical implementation challenges that may be barriers to adoption for many applications.

We observed that using two sensor locations (trunk IMU and pressure insoles) sacrificed minimal accuracy compared to using more sensor locations, e.g., all six distributed sensor locations, as shown in FIGS. 4A-4B and 6A-6B. This supports the idea that it may be possible to use a relatively small subset of sensors to make workplace implementation more practical, while still obtaining accurate estimates of back loading. These findings also demonstrate that more sensors, or more widely distributed sensors, should not be assumed to result in substantially more accurate musculoskeletal load monitoring tools. For monitoring lumbar loading during manual material handling, there appears to be a sweet spot for accuracy and practicality that involves using pressure insoles and a single IMU.

What Types of Algorithms Work Well for This Sensor Data Fusion? All the results presented here were developed using Gradient Boosted Decision Tree algorithms. We found this type of algorithm to work well during early exploration of the data. Within the Gradient Boosted Decision Tree framework, we utilized the histogram-based decision tree building algorithm, as it significantly reduces the training time with larger datasets (>10 k samples), but did not noticeably degrade prediction performance of the algorithms, compared to traditional Gradient Boosted Decision Trees. Using this approach, input signal values are separated into bins, reducing the computational complexity of splitting decisions and efficiently leveraging parallel computational resources.

In a pilot data analysis, we also explored other categories of algorithms/models, including generalized linear models, ensemble methods (random forests), shallow neural networks (2 hidden layers), and support vector regression. While most of these methods (linear, support vector regressions, forests) resulted in comparable prediction results to each other, Gradient Boosting consistently provided more accurate estimates in the preliminary data sets. Additionally, some of these methods (most notably, support vector regression) did not scale well with a large number of data points and became prohibitive to train.

We did not have success with traditional neural network models. This may have been because of insufficient number of layers, nodes, or the chosen activation functions. We note that the hyperparameter space for neural networks is significantly larger than for the other methods we tried. We provide this brief commentary on the explored set of machine learning algorithms for this problem domain to share the initial experiences. Our review and evaluation of alternative algorithm approaches is not exhaustive and there are certainly other applicable AI-based or statistical methods beyond this initial study. Such promising candidates include convolutional neural layers and recurrent neural networks, which may be interesting to explore in the future.

How Accurately Can Low Back Loading be Monitored during Manual Material Handling Tasks? The idealized results demonstrate the potential for a small number of sensors to provide accurate estimates of low back loading. Using a trunk IMU and pressure insoles resulted in lumbar moment estimates that were strongly correlated with lab-based lumbar moments (r²=0.89, FIG. 4B). This solution performed well across the broad range of tasks and lumbar moment magnitudes captured (FIG. 4A). The RMSE and MAPE accuracy results corroborated that this wearable sensor approach is very promising. The RMSE corresponds to less than 10% of the peak lumbar moments during heavy lifting. For context, we found that using just two sensor locations (trunk IMU and pressure insoles) during about 400 different material handling tasks exhibited similar levels of accuracy (r²=0.89 and RMSE=20 Nm) as those reported by Faber et al., which combined 8-17 IMUS and force-sensing shoes to estimate lumbar moments during 4 tasks that involved lifting and carrying a 10 kg box (r²=0.93 and RMSE<20 Nm).

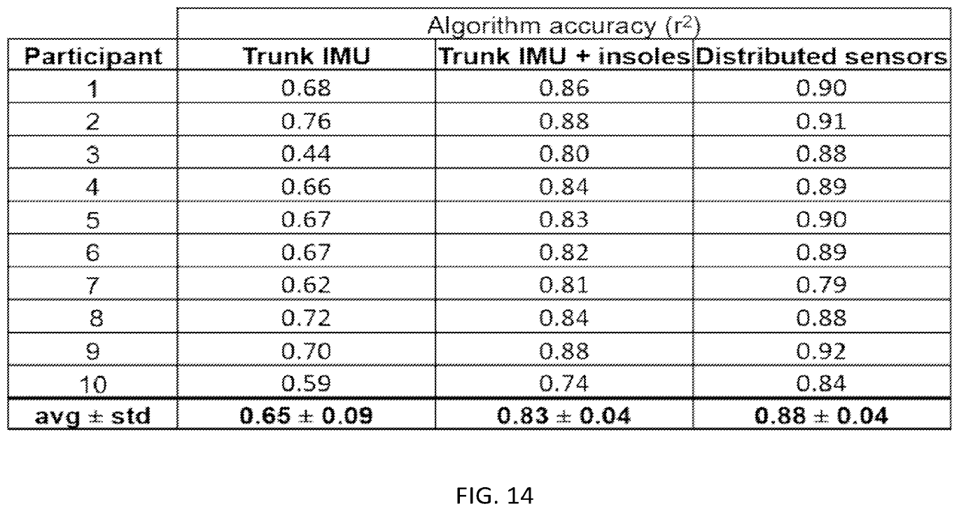

The real wearable results highlighted the technological key to realizing accurate estimates of back loading in the real world. Combining a real wearable trunk IMU and pressure insoles resulted in lower average accuracy than with the idealized wearable sensors (e.g., r²=0.80 vs. r²=0.89), and only marginal benefits over a real trunk IMU alone (r²=0.80 vs. r²=0.79). This appears to be due to variability in insole force estimates compared to vertical forces from idealized wearable sensors (i.e., from lab-based force plates, FIG. 14). In contrast, we found that trunk orientation from the real wearable sensor (trunk IMU) was a very strong indicator of trunk orientation from idealized wearable sensors (lab-based optical motion capture), with low variability, as shown in FIG. 14. Together, this seems to explain why GRFs were of similar importance as the trunk IMU when using the idealized wearable sensors, as shown in FIG. 3, but of much lower importance when using the real wearable sensors, as shown in FIG. 5. A key technological priority should be to reduce the variability in the insole force estimates. The good news is that there are various ways to improve these force estimates through advances in signal processing, calibrations, and sensor hardware, or via the optimization of sensors for pressure/force magnitudes expected in certain tasks such as material handling. As the variability in insole forces is reduced, the accuracy of algorithms developed using real wearable sensors will approach the accuracy observed using the idealized wearable sensors. We confirmed this to be true by replacing the real pressure insole data with idealized pressure insole data during algorithm development and finding lumbar moment estimates to have similar accuracy to the idealized wearable sensor algorithms. This replacement of the real pressure insole data with idealized data is an example of combining data related to object weight from a separate system (e.g., in this case from a separate measure of force under each foot, but it could alternatively be from data stored in a warehouse management system or other sensor system) with a single IMU (e.g., on the trunk) to accurately estimate musculoskeletal loading on the back.

These insights highlight the benefits of using idealized signals when exploring new wearable sensor solutions. If we used real wearable sensors alone, we may have concluded that pressure insoles do not improve back loading estimates compared to a single wearable trunk IMU. In actuality the pressure insoles provide unique and highly valuable force data, as shown in FIGS. 2 and 3, and Table 3, that can help distinguish when someone is lifting a heavy object vs. simply bending forward, and that can greatly improve capabilities for monitoring trends in low back loading (particularly at higher magnitudes). Overall, the complementary analyses, evaluating accuracies across a range of reduced sensor algorithms for both idealized and real wearable sensors, and ranking signal importances, provides a systematic and effective approach to identifying key sensor signals and promising wearable sensor combinations.

Benefits and Drawbacks of Single Wearable Sensor Solutions: The results demonstrate that a single IMU solution can perform reasonably well for estimating lumbar moments, as summarized in Table 3. The practical benefits includes relative simplicity for workplace implementation. The trunk IMU, and to a slightly lesser extent the thigh and pelvis IMUs, provided moderately high correlation coefficients up to r²=0.74 in idealized wearable sensor analysis, and up to r²=0.79 in the real wearable sensor analysis. The reason for the slightly stronger correlations with real wearable sensors for the trunk IMU algorithm is unknown, but may be due to a richer set of candidate signals that we input into the real vs. idealized wearable algorithms (see Table 1 vs. Table 2), which included additional spine segment and joint angle estimates from the Xsens functional skeleton calibration, and IMU accelerations and velocities. These results suggest that commercial wearables that place an IMU on one of these segments (trunk, pelvis, or thighs) are at least monitoring the types of signals that can be correlated with lumbar moments (with proper algorithm development and training).

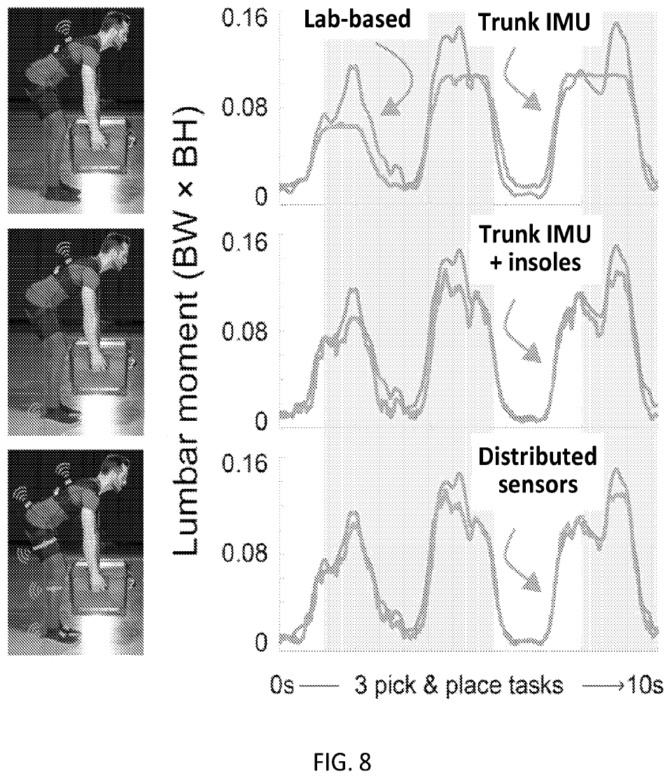

The critical drawback of single IMU wearables is that they fail to capture increases in lumbar loading when different objects are lifted, and as a result they tend to perform worse for higher lumbar moments, which unfortunately are the instances of highest ergonomic interest since these are most damaging to musculoskeletal tissues. This accuracy limitation at higher magnitudes is evident in plots of time series lumbar moments. For example, the trunk IMU algorithm does not capture the increase in low back loading peaks when a participant is picking and placing a 10 kg box (gray areas in FIG. 8). In contrast, these elevated back loads from the handheld mass are captured by solutions that use sensors at multiple locations that include pressure insoles along with at least one IMU, as shown in FIG. 8. The time-series plots show a representative lifting task, while the scatter plots and tables presented in the Results provide comprehensive results from all the participants and across all the manual material handling tasks collected.

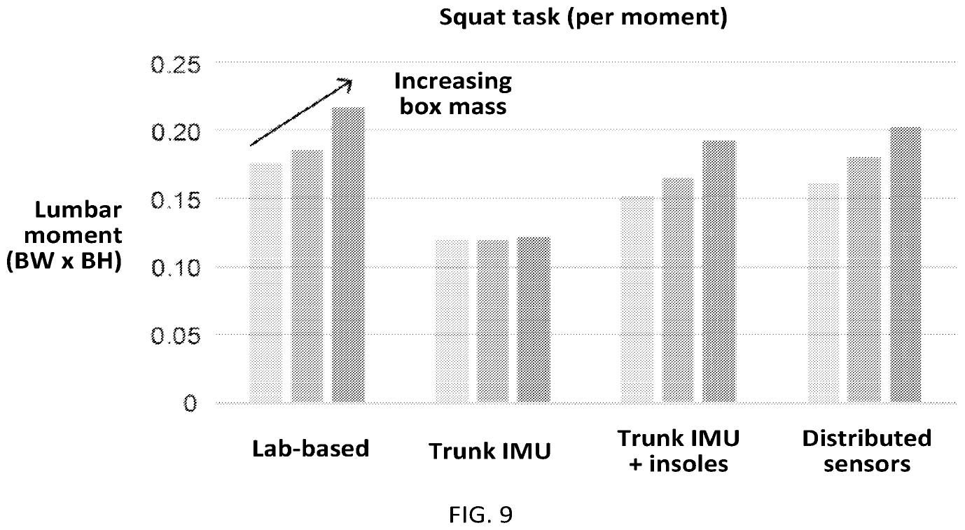

As another example, lifting objects of increasing mass with similar body posture causes an increase in peak lumbar moments, as shown in FIG. 9. Using a trunk IMU alone completely misses the trend of increasing low back loading when individuals adopt similar trunk orientations (i.e., postures) for each lift, while using a solution that includes both pressure insoles and an IMU captures these increasing back load trends, as shown in FIG. 9. These results confirm the expectations: while a single IMU (on the trunk, or elsewhere) may provide a reasonable estimate of back loading (or trends in loading) due to changes in general body posture, the estimation accuracy is compromised when objects of differing mass are handled or when other external forces are applied to the body (e.g., during pushing, pulling, or leaning). It may also be possible to use the trunk IMU plus pressure insoles combination during initial assessment of each worker, or intermittently over time, in order to better calibrate the trunk IMU (alone) for each worker—in effect supplementing the minimal single senor solution to improve accuracy and personalization. Of note, using the pressure insoles alone yielded fairly poor accuracy, as shown in FIG. 10, again highlighting the benefits of fusing data from multiple sensor locations.

In short, caution should be taken when using a single wearable (on anybody segment) to monitor low back loading, particularly in situations where external forces are variable, or when object masses being handled are not manually input (or otherwise accounted for) in algorithms. In general, observable body kinematics (e.g., positions, velocities, or accelerations from a single body segment) and directly measurable kinetics signals (e.g., ground reaction forces between the foot/shoe and ground) cannot be assumed to, and often do not, correlate with or estimate-well musculoskeletal loading on tissues inside the body. The use of a small number of sensors, and unique fusion of multi-sensor signals to non-invasively estimate musculoskeletal loading on the back is part of what distinguishes our novel approach. Further exploration is warranted to understand the implications of single IMU sensor accuracy within the context of risk assessment tool sensitivity, and to understand the validity of using single wearable sensor solutions for different types or subsets of manual material handling tasks. Wearable solutions that fuse data from multiple sensor locations (e.g., trunk IMU and pressure insoles) are expected to provide more accurate and reliable ways to automate ergonomic assessments or provide continuous daily risk monitoring for material handling jobs that involve lifting objects of varying weight; albeit with slightly more implementation complexity due to more sensor modalities, and presuming the variability in pressure insole force estimates can be adequately reduced.

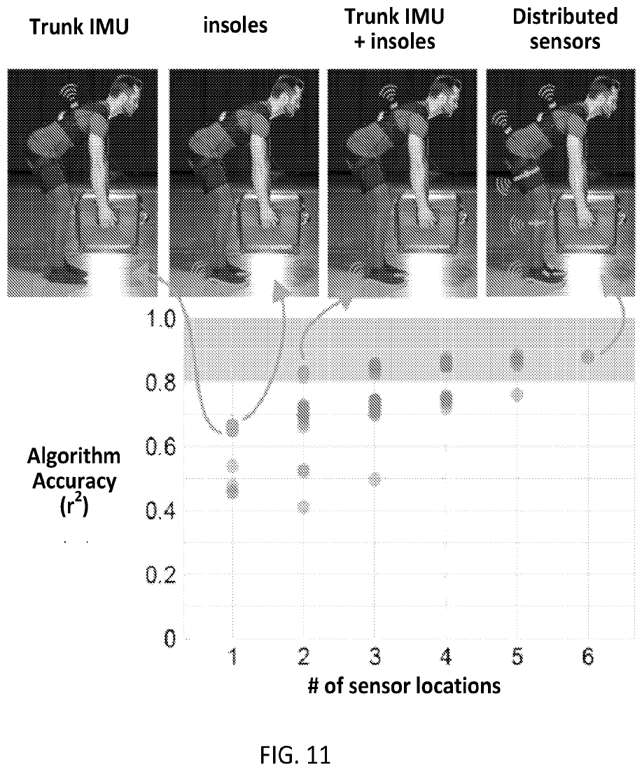

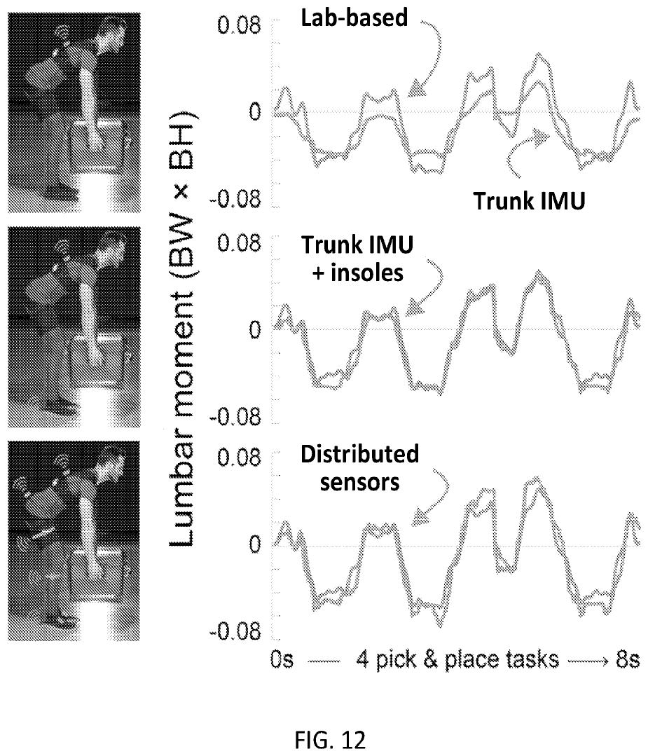

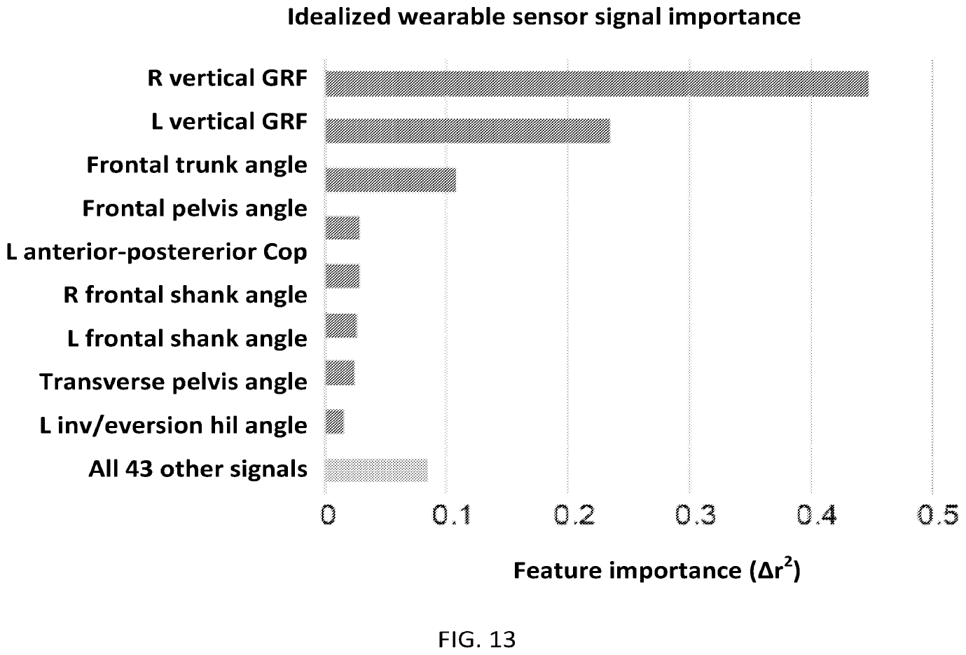

Lateral Bending Lumbar Moment Can Also Be Estimated with a Trunk IMU and Pressure Insoles: Lumbar extension moments have been shown to be a key metric for monitoring cumulative damage to the low back and resulting injury risk (see Introduction). However, there are also opportunities to provide a broader, multifactorial assessment of injury risk by monitoring other musculoskeletal loading metrics with wearables. One additional metric of interest to us was lumbar lateral bending moment, as increases in lateral bending moment contribute to increases in back muscle and disc compression forces, which influence cumulative damage to the low back. We therefore repeated the same algorithm development and evaluation process using the idealized wearable sensor data from this study, but using time series lumbar lateral bending moment as the target metric, as shown in FIGS. 11-14. Encouragingly, signals from the same set of wearable sensors (trunk IMU and pressure insoles) that were identified as most important for estimating lumbar extension moments were also the most important for estimating lateral bending moment, as shown in FIG. 13. Similar to our analysis of the lumbar extension moment, a single trunk IMU algorithm did not capture all trends in lateral bending moment, namely when the user held and moved objects of differing mass lateral to their body, as shown in FIG. 12. The trunk IMU algorithm resulted in an average accuracy of r²=0.65, as shown in FIG. 14. Combining the pressure insoles with the trunk IMU increased the accuracy to r²=0.83 (FIG. 14). This again demonstrates how a small set of wearable sensors (trunk IMU and pressure insoles) could provide a practical and accurate tool for monitoring low back loading (due to both lateral and extension moments), with only a relatively small reduction in accuracy compared to the full set of distributed sensors tested (r²=0.88, FIG. 14).

Limitations and Future Opportunities: Given the exploratory nature of developing next generation wearables, there were many interesting additional areas of research that were beyond the scope we chose to evaluate in this study. Numerous other candidate wearable sensors and emerging technologies, signal processing techniques, machine learning algorithms, and musculoskeletal metrics of interest could be explored in future studies. Additionally, while we focus on evaluating a tool for monitoring low back loading in a workplace environment, there are many other exciting research and clinical applications of a low back monitoring tool. For example, a similar wearable solution might be used in a clinical or home setting to monitor patients during post-injury or post-surgery rehabilitation, track their progress, or assist with return-to-work decisions.

Within the scope of this study, we note some limitations of the approach. First, the real wearable sensors used were research-grade instrumentation. Implementing algorithms on consumer-grade hardware, or any other hardware platform not tested here, would require additional algorithm calibration, validation and evaluation. Second, the number of participants tested was informed by our prior studies combining wearable sensors and machine learning, but this kind of exploratory (non-hypothesis-driven) research is not amenable to traditional sample size calculations. The consistency of results for individual participants using the k-fold validation analysis suggests that the sample was adequate, but we acknowledge that the understanding of how much data is enough to identify promising wearable monitoring tools using diverse machine learning techniques is continuing to evolve. Third, we did not use sensors to monitor the location of the object being lifted relative to the body (e.g., spine). Although this distance could be estimated by tracking multiple segments of the arms, we choose not to do this for reasons of simplicity and practicality. For now, adding this complexity seems unnecessary given that the simpler trunk IMU plus pressure insole solution presented here already shows strong potential for estimating lumbar moments. Fourth, we focused on load monitoring as a key risk factor for low back disorders, but it is worth reminding that sensors like the trunk IMU capture other data such as twisting (spine rotation) and trunk acceleration/deceleration, which can also be useful and complementary for injury risk assessment, for instance, by providing additional intermediate variables which can be input into load estimation algorithms or injury risk models. Fifth, we focused on how lumbar moments could be used as direct inputs into the LiFFT risk assessment tool. However, we should note that lumbar moments (and other wearable metrics like trunk orientation) could alternatively be input into other tools or models such as the NIOSH Lifting Equation. However, the NIOSH Lifting Equation requires some additional inputs such as a “Coupling” factor. Fully automating wearable risk assessment using the NIOSH Lifting Equation seems feasible, but would require some additional assumptions, modeling, and validation. Alternatively, lumbar moments computed here could be used with additional musculoskeletal modeling to estimate lumbar compression force or the forces on other targeted tissues in the back, such as by using computed muscle control or other load distribution algorithms. Sixth, algorithms were developed and evaluated on a broad range of movement tasks we identified as representative of many manual material handling jobs performed in workplace environments. The efficacy of using a trunk IMU and pressure insoles to monitor low back loading for other tasks or jobs outside of those tested would require additional validation and evaluation. To our knowledge, this is one of the largest databases ever collected from synchronized laboratory instrumentation and wearable sensors in this ergonomics and material handling domain. As such, we plan to use this dataset for future secondary analysis, and to make it available to other researchers interested in exploring additional research questions.

### CONCLUSIONS

Here, we present a promising wearable solution for the practical, automated, and accurate monitoring of low back loading during manual material handling. We found that two key sensors for accurately monitoring low back loading are a trunk IMU and pressure insoles. Using signals from these two sensors together with a Gradient Boosted Decision Tree algorithm has the potential to provide a practical (relatively few sensors), accurate (up to r²=0.89), and automated way (using wearables) to monitor time series lumbar moments across a broad range of material handling tasks. The trunk IMU could be replaced by thigh IMUS or a pelvis IMU without sacrificing much accuracy, but there was no practical substitute for the pressure insoles. The key to realizing accurate lumbar load estimates with this approach in the real world will be optimizing force estimates from pressure insoles. This promising wearable solution has the potential to transform low back injury risk assessment, monitoring, and prevention in various industries.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to activate others skilled in the art to utilize the invention and various embodiments and with various modifications as are suited to the particular use contemplated. Alternative embodiments will become apparent to those skilled in the art to which the invention pertains without departing from its spirit and scope. Accordingly, the scope of the invention is defined by the appended claims rather than the foregoing description and the exemplary embodiments described therein.

Some references, which may include patents, patent applications, and various publications, are cited and discussed in the description of the invention. The citation and/or discussion of such references is provided merely to clarify the description of the invention and is not an admission that any such reference is “prior art” to the invention described herein. All references cited and discussed in this specification are incorporated herein by reference in their entireties and to the same extent as if each reference was individually incorporated by reference.

### REFERENCES

[1]. U.S. Department of Labor. Back Injuries Prominent in Work-Related Musculoskeletal Disorder Cases in 2016. Available online: https://www.bls.gov/news.release/archives/osh_11092017.htm.
[2]. Luckhaupt, S. E.; Dahlhamer, J. M.; Gonzales, G. T.; Lu, M. L.; Groenewold, M.; Sweeney, M. H.; Ward, B. W. Prevalence, Recognition of Work-Relatedness, and Effect on Work of Low Back Pain Among U. S. Workers. Ann. Intern. Med. 2019, 171, 301-304.
[3]. Yang, H.; Haldeman, S.; Lu, M.-L.; Baker, D. Low Back Pain Prevalence and Related Workplace Psychosocial Risk Factors: A Study Using Data From the 2010 National Health Interview Survey. J. Manip. Physiol. Ther. 2016, 39, 459-472.
[4]. Gallagher, S.; Sesek, R. F.; Schall, M. C.; Huangfu, R. Development and validation of an easy-to-use risk assessment tool for cumulative low back loading: The Lifting Fatigue Failure Tool (LiFFT). Appl. Ergon. 2017, 63, 142-150.
[5]. Gallagher, S.; Schall, M. Musculoskeletal disorders as a fatigue failure process: Evidence, implications and research needs. Ergonomics 2016, 60, 255-269.
[6]. Edwards, W. B. Modeling Overuse Injuries in Sport as a Mechanical Fatigue Phenomenon. Exerc. Sport Sci. Rev. 2018.
[7]. Waters, T. R.; Lu, M.-L.; Occhipinti, E. New procedure for assessing sequential manual lifting jobs using the revised NIOSH lifting equation. Ergonomics 2007, 50, 1761-1770.
[8]. Faber, G. S.; Kingma, I.; Chang, C. C.; Dennerlein, J. T.; van Dieen, J. H. Validation of a wearable system for 3D ambulatory L5/S1 moment assessment during manual lifting using instrumented shoes and an inertial sensor suit. J. Biomech. 2020, 109671.
[9]. Conforti, I.; Mileti, I.; Panariello, D.; Caporaso, T.; Grazioso, S.; Del Prete, Z.; Lanzotti, A.; Di Gironimo, G.; Palermo, E. Validation of a novel wearable solution for measuring L5/S1 load during manual material handling tasks. In Proceedings of the 2020 IEEE International Workshop on Metrology for Industry 4.0 & IoT, Rome, Italy, 3-5 Jun. 2020; pp. 501-506.
[10]. Larsen, F. G.; Svenningsen, F. P.; Andersen, M. S.; de Zee, M.; Skals, S. Estimation of Spinal Loading During Manual Materials Handling Using Inertial Motion Capture. Ann. Biomed. Eng. 2020, 48, 805-821.
[11]. Koopman, S.; Kingma, I.; Faber, G. S.; Bornmann, J.; van Dieen, J. H. Estimating the L5S1 flexion/extension moment in symmetrical lifting using a simplified ambulatory measurement system. J. Biomech. 2018, 70, 242-248.
[12]. Matijevich, E. S.; Scott, L. R.; Volgyesi, P.; Derry, K. H.; Zelik, K. E. Combining wearable sensor signals, machine learning and biomechanics to estimate tibial bone force and damage during running. Hum. Mov. Sci. 2020, 74, 102690.
[13]. Li, J.; Wang, P.; Huang, H. J. Dry Epidermal Electrodes Can Provide Long-Term High Fidelity Electromyography for Limited Dynamic Lower Limb Movements. Sensors 2020, 20, 4848.
[14]. Colombini, D.; Occhipinti, E.; Alvarez-Casado, E.; Waters, T. R. Manual Lifting: A Guide to the Study of Simple and Complex Lifting Tasks; CRC Press: Boca Raton, Fla., USA, 2012.
[15]. Norman, R.; Wells, R.; Neumann, P.; Frank, J.; Shannon, H.; Kerr, M. A comparison of peak vs. cumulative physical work exposure risk factors for the reporting of low back pain in the automotive industry. Clin. Biomech. 1998, 13, 561-573.
[16]. Halilaj, E.; Rajagopal, A.; Fiterau, M.; Hicks, J. L.; Hastie, T. J.; Delp, S. L. Machine learning in human movement biomechanics: Best practices, common pitfalls, and new opportunities. J. Biomech. 2018, 81, 1-11.
[17]. Friedman, J. H. Stochastic gradient boosting. Comput. Stat. Data Anal. 2002, 38, 367-378.
[18]. Natekin, A.; Knoll, A. Gradient boosting machines, a tutorial. Front. Neurorobot. 2013, 7.
[19]. Ke, G.; Meng, Q.; Finley, T.; Wang, T.; Chen, W.; Ma, W.; Ye, Q.; Liu, T. Y. LightGBM: A Highly Efficient Gradient Boosting Decision Tree. In Advances in Neural Information Processing Systems 30; 2017; pp. 3146-3154.
[20]. Steel, R.; Torrie, J. Principles and Procedures of Statistics; McGraw-Hill: New York, N.Y., USA, 1960.
[21]. Matijevich, E. S.; Branscombe, L. M.; Scott, L. R.; Zelik, K. E. Ground reaction force metrics are not strongly correlated with tibial bone load when running across speeds and slopes: Implications for science, sport and wearable tech. PLoS ONE 2019, 14, e0210000.
[22]. Breiman, L. Random Forests. Mach. Learn. 2001, 45, 5-32.
[23]. Nemeth, G.; Ohlsen, H. Moment Arm Lengths of Trunk Muscles to the Lumbosacral Joint Obtained In Vivo with Computed Tomography. Spine 1986, 11, 158-160.
[24]. Guryanov, Histogram-Based Algorithm for Building Gradient Boosting Ensembles of Piecewise Linear Decision Trees. In Analysis of Images, Social Networks and Texts; Springer International Publishing: Cham, Switzerland, 2019; pp. 39-50.
[25]. Marras, W. S.; Granata, K. P. Spine loading during trunk lateral bending motions. J. Biomech. 1997, 30, 697-703.

1. A wearable device operably worn by a user for monitoring musculoskeletal loading on a back segment of the user, comprising:
a plurality of sensors, each sensor operably attached at a predetermined location of the user and configured to detect information about a biomechanical activity of a musculoskeletal system, wherein the plurality of sensors comprises at least one motion/orientation sensor; and
a processing unit in communication with the plurality of sensors and configured to process the detected information by the plurality of sensors to estimate the musculoskeletal loading and/or damage and/or injury risk, and communicate the estimated musculoskeletal loading and/or damage and/or injury risk to the user and/or a party of interest.

2. The wearable device of claim 1, wherein the plurality of sensors further comprises at least one pressure-sensing insole operably worn on at least one foot of the user.

3. The wearable device of claim 1, wherein the at least one motion/orientation sensor comprises an inertial measurement unit (IMU) operably attached to the trunk, pelvis, thighs, or shanks of the user.

4. The wearable device of claim 1, wherein the biomechanical activity of the musculoskeletal system comprises a segment orientation, a velocity or acceleration, and segmental load, wherein the segmental load comprises a location and/or magnitude of force or moment applied to the body segment.

5. The wearable device of claim 1, wherein the musculoskeletal loading comprises a lumbar moment, a lumbar spine or disc force, and/or a muscle, muscle group, tendon or ligament force.

6. The wearable device of claim 5, wherein the lumbar extension moment is used as a target musculoskeletal loading metric for estimating cumulative tissue damage and/or injury risk to the low back using a fatigue failure and/or finite element analysis.

7. The wearable device of claim 1, wherein the detected information by the plurality of sensors further includes information about the trunk or lumbar orientation/angles, or velocities, or accelerations, or frequency of lifting or bending movements, which are estimated or tracked, and then combined with or used in conjunction with the musculoskeletal loading estimates to estimate damage or assess injury risk.

8. The wearable device of claim 1, wherein the processing unit is configured to operably receive data of each object of which the user handles, acquired by a separate system, together with the detected information by the plurality of sensors, for processing the musculoskeletal loading and/or damage and/or injury risk, wherein the data of each object includes weight, size and location.

9. The wearable device of claim 8, wherein the separate system comprises an inventory management system that tracks the weight, size, or location of each object of which the user handles.

10. The wearable device of claim 8, wherein the separate system comprises a force-sensing crate handle or glove worn by the user that detects the force applied to each object of which the user handles.

11. The wearable device of claim 1, wherein the detected information by the plurality of sensors is processed by statistical modeling.

12. The wearable device of claim 11, wherein the statistical modeling comprises supervised model-based linear regression, decision trees or neural networks, and/or other data-driven machine learning or sensor fusion algorithms.

13. The wearable device of claim 12, wherein the statistical modeling comprises a gradient boosted decision tree algorithm.

14. The wearable device of claim 1, wherein the processing unit is further configured to estimate the musculoskeletal loading using reference data for calibrating or establishing a processing algorithm, wherein the reference data are either stored on data storage means in communication with the processing unit, or collected or inputted from a specific user.

15. The wearable device of claim 14, wherein the reference data are obtained by lab-based sensors, and the data storage means comprises a database, a cloud storage system, and/or a computer readable memory.

16. The wearable device of claim 1, wherein the processing unit is further configured to alert the user, via audio, visual or haptic feedback, when the musculoskeletal loading, damage, or injury risk is greater than a threshold that is predetermined or a threshold that is calibrated for a specific user.

17. The wearable device of claim 16, wherein the processing unit is further configured to advise the user on when and how to adjust their movements, actions or physical activity type and duration so as to reduce injury risks.

18. The wearable device of claim 1, wherein the processing unit is further configured to communicate to a computer, a smartphone, a smartwatch, a tablet or other user feedback or data acquisition device for inputting user inputs, and outputting at least one of the estimated musculoskeletal loading, alert and advice, estimates of damage or damage accumulation, and/or probability of fracture or injury risk, and storing the estimated musculoskeletal loading, alert and advice, estimates of damage or damage accumulation, and/or probability of fracture or injury risk.

19. The wearable device of claim 1, further comprising a biofeedback unit in communication with the processing unit for outputting and/or displaying at least one of the estimated musculoskeletal loading, alert and advice, estimates of damage or damage accumulation, and/or probability of fracture or injury risk using audible, visual, tactile, haptic, thermal, electrical or other biofeedback means, and storing the estimated musculoskeletal loading, alert and advice, estimates of damage accumulation, and/or probability of fracture or injury risk.

20. The wearable device of claim 19, wherein the biofeedback unit comprises a user interface device for user inputs.

21. The wearable device of claim 20, wherein the user inputs comprise height, weight, body mass index, age, gender, diet, training schedule, subjective pain/fatigue, bone cross-sectional area, bone geometry, bone density, bone composition, GPS position, altitude of the user and/or other personal health or demographic data.

22. The wearable device of claim 1, wherein the information further comprises data acquired from additional sensors that monitor sleep patterns, heart rate, heart rate variability, rest time between physical activity or other markers of tissue rest or remodeling, or physiological recovery.

23. The wearable device of claim 1, wherein the damage is estimated by summing across load metrics taken to an exponential power.

24. The wearable device of claim 1, wherein the plurality of sensors is combined with or integrated into an exoskeleton, exosuit, smart clothing, or other wearable assistance device.

25. The wearable device of claim 24, wherein the plurality of sensors onboard the exoskeleton, exosuit, smart clothing or other wearable assistance device are used to estimate contributions to lumbar loading, and then these estimates are used in calculations of musculoskeletal loading and/or damage and/or injury risk on the back or other body segments, wherein the moment from exoskeleton is subtracted from total lumbar moment to estimate moment borne by biological tissues.

26. The wearable device of claim 24, wherein the musculoskeletal loading is used for control or evaluation of the exoskeleton, exosuit, smart clothing or other wearable assistance device.

27. The wearable device of claim 26, wherein a reinforcement learning algorithm incrementally learns optimal control of the exoskeleton, exosuit, smart clothing or other wearable assistive device from wearable sensor inputs based on real-time feedback from the user and previously observed motion trajectories.

28. The wearable device of claim 1, wherein a state machine is used to identify specific activities, and then different algorithms are used to process information and to estimate the musculoskeletal loading and/or damage and/or injury risk depending on the current state.

29. The wearable device of claim 1, wherein the estimates of the musculoskeletal loading and/or damage and/or injury risk are computed via real-time or near-real-time estimation algorithms.

30. The wearable device of claim 1, wherein the estimated musculoskeletal loading and/or damage and/or injury risk is communicated to the user and/or a party of interest via one or more wireless or wired communication interfaces, either in real-time, near-real-time or at a later time.

31. A method for monitoring musculoskeletal loading on a back segment of a user wearing a wearable device including a plurality of sensors that is temporally and/or spatially synchronized to each other, each sensor worn by the user at a predetermined location, wherein the plurality of sensors comprises at least one motion/orientation sensor, comprising:
receiving information about a biomechanical activity of a musculoskeletal system from the plurality of sensors;
estimating musculoskeletal loading and/or damage and/or injury risk of the back segment based on the received information from the plurality of sensors; and
communicating the estimated musculoskeletal loading and/or damage and/or injury risk to the user and/or a party of interest.

32. The method of claim 31, wherein the plurality of sensors further comprises at least one pressure-sensing insoles operably worn on at least one foot of the user.

33. The method of claim 31 or 30, wherein the at least one motion/orientation sensor comprises an inertial measurement unit (IMU) operably attached to the trunk, pelvis, thighs, or shanks of the user.

34. The method of claim 31, wherein the estimating step is performed by statistical modeling.

35. The method of claim 34, wherein the statistical modeling comprises supervised model-based linear regression, decision trees or neural networks, and/or other data-driven machine learning or sensor fusion algorithms.

36. The method of claim 35, wherein the statistical modeling comprises a gradient boosted decision tree algorithm.

37. The method of claim 31, wherein the estimating step computes the musculoskeletal loading using reference data to calibrate or establish the processing algorithm, so as to determine a condition of the body structure based on the estimated musculoskeletal loading, the condition including a normal condition or a graduated risk of injury.

38. The method of claim 37, wherein the reference data are obtained by lab-based sensors.

39. The method of claim 31, wherein the communicating step comprises inputting user inputs, and outputting at least one of the estimated musculoskeletal loading, alert and advice, estimates of damage or damage accumulation, and/or probability of fracture or injury risk, and storing the estimated musculoskeletal loading, alert and advice, estimates of damage or damage accumulation, and/or probability of fracture or injury risk.

40. The method of claim 39, wherein the communicating step comprises advising the user on when and how to adjust their movements, actions or physical activity type and duration so as to reduce injury risks.

41. The method of claim 31, wherein the plurality of sensors is combined with or integrated into an exoskeleton, exosuit, smart clothing, or other wearable assistance device.

42. The method of claim 41, further comprising controlling or evaluating the exoskeleton, exosuit, smart clothing or other wearable assistance device using the musculoskeletal loading.

43. The method of claim 42, wherein the control of the exoskeleton, exosuit, smart clothing or other wearable assistive device is optimized from wearable sensor inputs based on real-time feedback from the user and previously observed motion trajectories, using a reinforcement learning algorithm.

44. The method of claim 31, further comprising identifying specific activities using a state machine, and processing information and to estimate the musculoskeletal loading and/or damage and/or injury risk with different algorithms depending on the current state.

45. The method of claim 31, wherein the estimates of the musculoskeletal loading and/or damage and/or injury risk are computed via real-time or near-real-time estimation algorithms.

46. The method of claim 31, wherein the estimated musculoskeletal loading and/or damage and/or injury risk is communicated to the user and/or a party of interest via one or more wireless or wired communication interfaces, either in real-time, near-real-time or at a later time.

47. The method of claim 31, wherein the biomechanical activity of the musculoskeletal system comprises a segment orientation, a velocity or acceleration, and a segmental load, wherein the segmental load comprises a location and/or magnitude of force or moment applied to the body segment.

48. A non-transitory computer-readable medium storing computer executable instructions to operate a wearable device for monitoring musculoskeletal loading on a back segment of a user according to the method of claim 31.
